(19) United States
(12) Patent Application Publication
(10) Pub. No.: US 2026/0069895 A1
(43) Pub. Date: Mar. 12, 2026

(54) METHODS AND SYSTEMS OF IMPROVING MEDICAL CONDITIONS VIA ULTRASOUND NEUROMODULATION OF THE AUTONOMIC NERVOUS SYSTEM

(71) Applicant: West Virginia University Board of Governors on behalf of West Virginia University, Morgantown, WV (US)

(72) Inventors: Ali Rezai, Morgantown, WV (US); Victor Finomore, Morgantown, WV (US)

(21) Appl. No.: 19/324,780

(22) Filed: Sep. 10, 2025

Related U.S. Application Data
(60) Provisional application No. 63/693,313, filed on Sep. 11, 2024.

(51) Int. Cl. A61N 7/00

(52) U.S. Cl. CPC A61N 7/00; A61N 2007/0021

(57) ABSTRACT

Methods and systems for improving medical conditions by delivering a focused ultrasound signal to target sites of the autonomic nervous system. The focused ultrasound signal can have a mechanical index of between about 0.1 to about 5.0 and/or an acoustic pressure of between about 0.1 MPa to about 2.5 MPa. The medical condition can comprise chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence or binge eating.

### RELATED APPLICATION

This application claims priority from U.S. Application No. 63/693,313, filed 11 Sep. 2024. The subject matter of this application is incorporated herein by reference in its entirety.

### TECHNICAL FIELD

The present technology is directed to methods and systems for improving medical conditions by delivering a focused ultrasound signal (FUS) to a neural target site of the autonomic nervous system (ANS).

### BACKGROUND

Neuromodulation is a series of techniques that can alter nerve activity in the body to normalize nervous tissue function. It can involve applying stimuli, such as electrical stimulation or chemical agents, to specific neurological sites. Neuromodulation can help decrease pain and increase mobility by changing the way nerves carry information to and from the brain. Neuromodulation can be used to treat a wide range of conditions involving nerves, muscles, and brain function. For example, transcutaneous electrical nerve stimulation (TENS) units and implanted spinal cord stimulators are based on the concept of neuromodulation and can provide substantial relief for many patients. Neuromodulation can also be used to treat treatment-resistant depression.

### SUMMARY

In an aspect, a method of improving a medical condition in a patient in need thereof is provided. The method can comprise delivering a focused ultrasound neuromodulation signal to a neural target site of the patient's autonomic nervous system. The ultrasound signal can have a mechanical index of between about 0.1 to about 5.0 and/or an acoustic pressure of between about 0.1 MPa to about 2.5 MPa, the medical condition comprising chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence or binge eating.

In another aspect, a computer program is provided comprising instructions, which, when the program is executed by a processor causes the steps of controlling an ultrasound pulse generator to deliver a focused ultrasound neuromodulation signal to a neural site of the autonomic nervous system, the ultrasound signal having a mechanical index of between about 0.1 to about 5.0 and/or an acoustic pressure of between about 0.1 MPa to about 2.5 MPa and to a site of the ANS sufficient to improve chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence, or binge eating.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a system for providing focused ultrasound treatment for treatment or diagnosis of a disorder to a specific region of interest;

FIG. 2 represents a method for screening a patient for a specific disorder;

FIG. 3 illustrates a method for using cues to generate features used to screen patients for a disorder;

FIG. 4 illustrates one method for monitoring a patient to predict or detect an onset of symptoms associated with a disorder for a patient;

FIG. 5 illustrates another example of a method for evaluating a patient for an onset of symptoms requiring treatment via focused ultrasound treatment;

FIG. 6 illustrates another method for targeting focused ultrasound treatment in a for treatment of a disorder using acute feedback; and

FIG. 7 illustrates a method for selecting a dose for focused ultrasound treatment.

### DETAILED DESCRIPTION

The present disclosure relates to methods and systems for improving medical conditions via ultrasound neuromodulation of the ANS. In an aspect, a method of improving a medical condition in a patient in need thereof can comprise delivering a focused ultrasound neuromodulation signal to a neural target site of the patient's autonomic nervous system, the ultrasound signal having an mechanical index of between about 0.1 and about 5 and/or an acoustic pressure of between about 0.1 MPa and about 2.5 MPa. The focused ultrasound neuromodulation signal can be ablative or non-ablative. The medical condition can comprise chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence, or binge eating. In certain aspects, a clinical or feedback parameter (as disclosed in more detail below) is used to determine whether to apply and/or adjust delivery ultrasound therapy.

In another aspect, a computer program is provided that comprises instructions, which, when the program is executed by processor a causes the steps of controlling an ultrasound pulse generator to dli a focused ultrasound neuromodulation signal to a neural site of the autonomic nervous system. The ultrasound signal can have a mechanical index of between about 1 and about 5 and/or an acoustic pressure of between about 1 MPa and about 2.5 MPa. The ultrasound signal is delivered to a site of the ANS sufficient to improve chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex-regional pain syndrome, post-traumatic stress disorder (PTSD), anxiety and anxiety-associated disorders, autism, fibromyalgia, uterine function, metabolic disorder, urinary incontinence, or binge eating. In certain aspects, the processor can apply or adjust application of the ultrasound signal based in clinical/feedback parameters disclosed in more detail below.

As used herein with respect to a described element, the terms “a,” “an,” and “the” include at least one or more of the described element(s) including combinations thereof unless otherwise indicated. Further, the terms “or” refer to “and/or” and combinations thereof unless otherwise indicated. By “substantially,” “approximately, or “about” is meant that the shape, size, configuration or value of the described element need not have the mathematically exact described shape, size, configuration or value of the described element but can have a shape, size, configuration or value that is recognizable by one skilled in the art as generally or approximately having the described shape, size, configuration, or value of the described element. As such “substantially,” “approximately,” or “about” refers to the complete or nearly complete extent of a characteristic, property, state, structure or value. The exact allowable degree of deviation from the characteristic, property, state, structure, or value will be so as to have the same overall result as if the absolute characteristic, property, state, structure, or value were obtained. The terms “first,” “second,” etc. are used to distinguish one element from another and not used in a quantitative sense unless indicated otherwise. Thus, a “first” element described below could also be termed a “second” element. A “patient” as used herein is a mammal such as, for example, a human being, dog, cat, horse, pig, sheep, cow, or other domesticated animal.

As used herein, a “clinical parameter” is any value representing a patient that is relevant to the patients' risk of a disorder or a progression of a disorder. Clinical parameters can include values measured by clinicians in a clinical environment, values measured outside of a clinical environment by one or more wearable or portable devices, or values retrieved from an electronic health records (EHR) interface and/or other available databases. It will be appreciated that the clinical parameter can also be referred to herein as “feedback parameter” including, for example, a physiological, cognitive, behavioral, psychosocial or anatomical parameter associated with the patient.

As used herein, a “predictive model” is a mathematical model or machine learning model that either predicts a future state of a parameter or estimates a current state of a parameter that cannot be directly measured. A predictive model is trained on training samples, each training sample comprising a set of values used for predicting the parameter in the predictive model and a known value for the parameter. It will be appreciated that a predictive model can be implemented as machine executable instructions.

As used herein, a “categorical value” is a value that can be represented by one of a number of discrete possibilities, which may or may not have a meaningful ordinal ranking. A “continuous value,” as used herein, is a value that can take on any of a number of numerical values within a range. It will be appreciated that, in a practical application, values are expressed in a finite number of significant digits, and thus a “continuous value” can be limited to a number of discrete values within the range.

As used herein, data is provided from a first system to a second system when it is either provided directly from the first system to the second system, for example, via a local bus connection, or stored in a local or remote non-transitory memory by the first system for later retrieval by the second system. Accordingly in some implementations, the first system and the second system can be located remotely and in communication only through an intermediate medium connected to at least one of the systems by a network connection.

“Registration” of two or more images includes any process that assigns relative locations between pixels or multi-pixel features across two or more images. This assignation can be represented, for example, via an explicit transformation model between two or more images or via feature matching techniques that identify common structural features across two images.

An “average,” as used herein, can be any measure of central tendency, including but not limited to, an arithmetic mean, a geometric mean, a median, and a mode. It will be appreciated that, where a mean for a set of values is used as the average, the mean can be taken from a subset of the set of values to eliminate outliers within the set of values. For example, values between the fifth and the ninety-fifth percentile can be used to generate the mean.

An “intensity profile,” as used herein, represents a spatial variation in the intensity of localized energy provided to a target. An intensity profile can include a variance between two sides of a region provided with the localized energy or a more complex spatial variation of the energy.

### Autonomic Nervous System

The nervous system is divided into the somatic nervous system and the ANS. In general, the somatic nervous system controls organs under voluntary control (e.g., skeletal muscles) and the ANS controls individual organ function and homeostasis. For the most part, the ANS is not subject to voluntary control. The ANS is also referred to as the visceral or automatic system. The ANS can be viewed as a “real-time” regulator of physiological functions that extracts features from the environment and, based on that information, allocates an organism's internal resources to perform physiological functions for the benefit of the organism, e.g., responds to environment conditions in a manner that is advantageous to the organism.

The ANS conveys sensory impulses to and from the central nervous system to various structures of the body such as organs and blood vessels, in addition to conveying sensory impulses through reflex arcs. For example, the ANS controls: constriction and dilatation of blood vessels; heart rate; the force of contraction of the heart; contraction and relaxation of smooth muscle in various organs; lungs; stomach; colon; bladder; and visual accommodation, secretions from exocrine and endocrine glands, etc. The ANS does this through a series of nerve fibers, and more specifically through efferent and afferent nerves. The ANS acts through a balance of its two components: the sympathetic nervous system (SNS) and the parasympathetic nervous system (PSNS), which are two anatomically and functionally distinct systems. Both of these systems include myelinated preganglionic fibers, which make synaptic connections with unmyelinated postganglionic fibers, and it is these fibers that then innervate the effector structure. These synapses usually occur in clusters called ganglia. Most organs are innervated by fibers from both divisions of the ANS, and the influence is usually opposing (e.g., the vagus nerve slows the heart, while the sympathetic nerves increase its rate and contractility), although it may be parallel (e.g., as in the case of the salivary glands).

The PSNS is the part of the ANS controlling a variety of autonomic functions including, but not limited to, involuntary muscular movement of blood vessels and gut and glandular secretions from eye, salivary glands, bladder, rectum and genital organs. The vagus nerve is part of the PNS. Parasympathetic nerve fibers are contained within the last five cranial nerves and the last three spinal nerves and terminate at parasympathetic ganglia near or in the organ they supply. The actions of the PNS are broadly antagonistic to those of the SNS; lowering blood pressure, slowing heartbeat, stimulating the process of digestion etc. The chief neurotransmitter in the PSNS is acetylcholine. Neurons of the parasympathetic nervous system emerge from the brainstem as part of the Cranial nerves III, VII, IX and X (vagus nerve) and also from the sacral region of the spinal cord via Sacral nerves. Because of these origins, the PNS is often referred to as the “craniosacral outflow”.

In the PSNS, both pre- and post-ganglionic neurons are cholinergic (i.e., they utilize the neurotransmitter acetylcholine). Unlike adrenaline and noradrenaline, which the body takes around 90 minutes to metabolize, acetylcholine is rapidly broken down after release by the enzyme cholinesterase. As a result, the effects are relatively brief in comparison to the SNS. Each pre-ganglionic parasympathetic neuron synapses with just a few post-ganglionic neurons, which are located near, or in, the effector organ, a muscle or gland. As noted above, the primary neurotransmitter in the PSNS is acetylcholine such that acetylcholine is the neurotransmitter at all the pre-ganglionic neurons and many of the post-ganglionic neurons of the PSNS. Some of the post-ganglionic neurons, however, release nitric oxide as their neurotransmitter.

The SNS is the part of the ANS comprising nerve fibers that leave the spinal cord in the thoracic and lumbar regions and supply viscera and blood vessels by way of a chain of sympathetic ganglia running on each side of the spinal column, which communicate with the central nervous system via a branch to a corresponding spinal nerve. The SNS controls a variety of autonomic functions including, but not limited to, control of movement and secretions from viscera and monitoring their physiological state, stimulation of the sympathetic system inducing, e.g., the contraction of gut sphincters, heart muscle and the muscle of artery walls, and the relaxation of gut smooth muscle and the circular muscles of the iris. The chief neurotransmitter in the SNS is adrenaline, which is liberated in the heart, visceral muscle, glands and internal vessels, with acetylcholine acting as a neurotransmitter at ganglionic synapses and at sympathetic terminals in skin and skeletal muscles. The actions of the SNS tend to be antagonistic to those of the PSNS.

### Focused Ultrasound

Ultrasound can be defined as an acoustic wave above 20 kHz, beyond the frequency range of human hearing. In a typical neuromodulation procedure, a pulse generator emits an electrical waveform, which is amplified and carried to an ultrasound transducer housing a piezoelectrical element coupled to its active face. An ultrasound transducer and ultrasound device are used interchangeably herein. The amplified electrical signal excites the piezoelectric element, which mechanically oscillates the active face of the transducer.

### FUS Parameters

Table I provides examples of FUS/sonication parameters that can be measured.

TABLE I
Sonication parameters.

| | Abbrev. | |
|---|---|---|
| Parameter | (Unit) | Description |
| Ultrasound | f₀ (kHz or | The frequency of ultrasonic pressure |
| operating | MHz) | waves; the carrier frequency of the |
| frequency | | ultrasound signal |
| Pulse | PD (ms) | The shortest continuous period of |
| duration | | sonication. When applying continuous |
| | | TUS, pulse duration is equal to the |
| | | pulse train duration; the duration of a |
| | | pulse in a series of ultrasound pulses |
| Pulse | PRF (Hz) | The frequency of pulse delivery within |
| repetition | | the pulse train duration; multiplicative |
| frequency/ | | inverse of the pulse repetition interval |
| rate | | |
| Duty cycle | DC (%) | The percentage of time sonication is |
| | | delivered during the pulse repetition |
| | | interval. The duty cycle is only defined |
| | | for rectangular pulses. (or pulse train |
| | | duration) |
| Spatial | pₛₚ | The position at which the ultrasound |
| peak/spatial | | signal has a highest amplitude. The |
| peak | | spatial peak occurs within a focal spot |
| pressure | | of the ultrasound transducer; The |
| | | amplitude of the pressure during the |
| | | steady part of the pulse at the location |
| | | of the spatial peak. |
| Spatial peak | ISPPA | The intensity of the pulse at the spatial |
| pulse | (W/cm2) | peak (focal peak) time averaged over |
| average | | the pulse duration. |
| intensity | | |
| Spatial peak | I_{SPTA} | The intensity of the ultrasonic stimulus |
| temporal | (mW/cm²) | at the spatial peak time averaged over |
| average | | a defined period of time (i.e., across the |
| intensity | | stimulus duration). The temporal |
| | | window must be defined. |
| Mechanical | MI | A unitless index for assessing the |
| Index | | likelihood of cavitational bioeffects. |
| | | Where pr.3 is the pressure derated using |
| | | αα = 0.3 dB cm⁻¹ MHz⁻¹ |
| Acoustic | Megapascal | Amount of force applied per unit area |
| Pressure | (MPa) | and is typically thought of as a local |
| | | pressure deviation relative to the |
| | | ambient surround pressure |
| Power | Watts | The power of the electrical waveform |
| | | emitted by ultrasound pulse generator |
| | | which is amplified and carried to one or |
| | | more ultrasound transducers |

Z = Acoustic Impedance

In further detail, to characterize the intensity of a pulsing protocol, a needle hydrophone can be used to measure the instantaneous pressure (Pᵢ) in Megapascals applied by the transducer in an acoustic medium. The instantaneous intensity (Iᵢ) is proportional to the square of the instantaneous pressure, and inversely related to the density (p) and speed of sound (c) in the propagating medium. The pulse intensity integral (PII) is then derived by integrating the instantaneous intensity over the duration of the pulse. From the PII, two measures of acoustic exposure can be derived: the spatial-peak temporal average (I_{SPTA}) and the spatial-peak pulse average (I_{SPPA}). I_{SPTA} measures the average intensity during the entire sonication and scales in proportion to sonication duration. Conversely, I_{SPPA} represents the average intensity over a single pulse, providing an estimate of short-term mechanical bioeffects. Another parameter is acoustic pressure, which is the amount of force applied per unit area and is typically thought of as a local pressure deviation relative to the ambient surround pressure. It is commonly represented with the symbol p and is given in Megapascal (MPa) units. Another parameter that can be measured is the mechanical index (MI), a unitless measure which is equal to peak negative pressure divided by the square of the fundamental frequency. The MI can estimate the risk of potentially destructive biomechanical effects on tissues, such as inertial cavitation. Frequency can also be measured, which is the number of wave cycles per second and is determined by the rate at which the ultrasound source oscillates and influences many aspects of tissue interaction. It is commonly represented with the symbol f and is given in Megahertz (MHz) units.

### FUS Parameter Values

The focused ultrasound signal can be generated with suitable stimulation parameters. In one implementation, focused ultrasound signal is generated by the ultrasound pulse generator with a power having a range that is one of the following: 10-20 W, 20-30 W, 30-40 W, 40-50 W, 50-60 W, 60-70 W, 70-80 W, 80-90 W, 90-100 W, 100-110 W, 110-120 W, 120-130 W, 130-140 W, 140-150 W, 10-30 W, 20-40 W, 30-50 W, 40-60 W, 50-70 W, 60-80 W, 70-90 W, 80-100 W, 90-110 W; 100-120 W, 110-130 W, 120-140 W, 130-150 W, 10-40 W, 20-50 W, 30-60 W, 40-70 W, 50-80 W, 60-90 W, 70-100 W; 80-110 W, 90-120 W, 100-130 W, 110-140 W, 120-150 W, 10-50 W, 20-60 W, 30-70 W, 40-80 W, 50-90 W, 60-100 W, 70-110 W, 80-120 W, 90-130 W, 100-140 W, 110-150 W, 10-60 W, 20-70 W, 30-80 W, 40-90 W, 50-100 W, 60-110 W, 70-120 W, 80-130 W, 90-140 W, 100-150 W, 10-70 W, 20-80 W, 30-90 W, 40-100 W, 50-110 W, 60-120 W, 70-130 W; 80-140 W, 90-150 W, 10-80 W, 20-90 W, 30-100 W, 40-110 W, 50-120 W, 60-130 W, 70-140 W, 80-150 W, 10-90 W, 20-100 W, 30-110 W, 40-120 W, 50-130 W, 60-140 W, 70-150 W, 10-100 W, 20-110 W, 30-120 W, 40-130 W, 50-140 W, 60-150 W, 10-110 W, 20-120 W, 30-130 W, 40-140 W, 50-150 W, 10-120 W, 20-130 W, 30-140 W, 40-150 W, 10-130 W; 20-140 W, 30-150 W, 10-140 W, 20-150 W, and 10-150 W.

In another example, focused ultrasound is provided with a mechanical index between 0.1 and five. In a further example, focused ultrasound is provided with a mechanical index between one and five. Specifically, focused ultrasound treatment is provided with a mechanical index between any of 1-1.2, 1.1-1.3, 1.2-1.4, 1.3-1.5, 1.4-1.6, 1.5-1.7, 1.6-1.8, 1.7-1.9, 1.8-2, 1.9-2.1, 2-2.2, 2.1-2.3, 2.2-2.4, 2.3-2.5, 2.4-2.6, 2.5-2.7, 2.6-2.8, 2.7-2.9, 2.8-3, 2.9-3.1, 3-3.2, 3.1-3.3, 3.2-3.4, 3.3-3.5, 3.4-3.6, 3.5-3.7, 3.6-3.8, 3.7-3.9, 3.8-4, 3.9-4.1, 4-4.2, 4.1-4.3, 4.2-4.4, 4.3-4.5, 4.4-4.6, 4.5-4.7, 4.6-4.8, 4.7-4.9, 4.8-5, 1-1.4, 1.1-1.5, 1.2-1.6, 1.3-1.7, 1.4-1.8, 1.5-1.9, 1.6-2, 1.7-2.1, 1.8-2.2, 1.9-2.3, 2-2.4, 2.1-2.5, 2.2-2.6, 2.3-2.7, 2.4-2.8, 2.5-2.9, 2.6-3, 2.7-3.1, 2.8-3.2, 2.9-3.3, 3-3.4, 3.1-3.5, 3.2-3.6, 3.3-3.7, 3.4-3.8, 3.5-3.9, 3.6-4, 3.7-4.1, 3.8-4.2, 3.9-4.3, 4-4.4, 4.1-4.5, 4.2-4.6, 4.3-4.7, 4.4-4.8, 4.5-4.9, 4.6-5, 1-1.8, 1.1-1.9, 1.2-2, 1.3-2.1, 1.4-2.2, 1.5-2.3, 1.6-2.4, 1.7-2.5, 1.8-2.6, 1.9-2.7, 2-2.8, 2.1-2.9, 2.2-3, 2.3-3.1, 2.4-3.2, 2.5-3.3, 2.6-3.4, 2.7-3.5, 2.8-3.6, 2.9-3.7, 3-3.8, 3.1-3.9, 3.2-4, 3.3-4.1, 3.4-4.2, 3.5-4.3, 3.6-4.4, 3.7-4.5, 3.8-4.6, 3.9-4.7, 4-4.8, 4.1-4.9, 4.2-5, 1-2, 1.3-2.3, 1.5-2.5, 1.7-2.7, 2-3, 2.3-3.3, 2.5-3.5, 2.7-3.7, 3-4, 3.3-4.3, 3.5-4.5, 3.7-4.7, 4-5, 1-2.5, 1.5-3, 2-3.5, 2.5-4, 3-4.5, 3.5-5, 1-3, 1.5-3.5, 2-4, 2.5-4.5, 3-5, 1-3.5, 1.5-4, 2-4.5, 2.5-5, 1-4, 1.5-4.5, and 2-5. In certain aspects, the mechanical index is about 2.7, about 4.2 or less than about 0.8.

In one implementation, focused ultrasound treatment is provided with a pulse duration between 3 ms and 200 ms. In another implementation, focused ultrasound treatment is provided with a pulse duration between any of 10-20 ms, 20-30 ms, 30-40 ms, 40-50 ms, 50-60 ms, 60-70 ms, 70-80 ms, 80-90 ms, 90-100 ms, 100-110 ms, 110-120 ms, 120-130 ms, 130-140 ms, 140-150 ms, 150-160 ms, 160-170 ms, 170-180 ms, 180-190 ms, 190-200 ms, 10-30 ms, 20-40 ms, 30-50 ms, 40-60 ms, 50-70 ms, 60-80 ms, 70-90 ms, 80-100 ms, 90-110 ms, 100-120 ms, 110-130 ms, 120-140 ms, 130-150 ms, 140-160 ms, 150-170 ms, 160-180 ms, 170-190 ms, 180-200 ms, 10-40 ms, 20-50 ms, 30-60 ms, 40-70 ms, 50-80 ms, 60-90 ms, 70-100 ms, 80-110 ms, 90-120 ms, 100-130 ms, 110-140 ms, 120-150 ms, 130-160 ms, 140-170 ms, 150-180 ms, 160-190 ms, 170-200 ms, 10-50 ms, 20-60 ms, 30-70 ms, 40-80 ms, 50-90 ms, 60-100 ms, 70-110 ms, 80-120 ms, 90-130 ms, 100-140 ms, 110-150 ms, 120-160 ms, 130-170 ms, 140-180 ms, 150-190 ms, 160-200 ms, 10-60 ms, 20-70 ms, 30-80 ms, 40-90 ms, 50-100 ms, 60-110 ms, 70-120 ms, 80-130 ms, 90-140 ms, 100-150 ms, 110-160 ms, 120-170 ms, 130-180 ms, 140-190 ms, 150-200 ms, 10-70 ms, 20-80 ms, 30-90 ms, 40-100 ms, 50-110 ms, 60-120 ms, 70-130 ms, 80-140 ms, 90-150 ms, 100-160 ms, 110-170 ms, 120-180 ms, 130-190 ms, 140-200 ms, 10-80 ms, 20-90 ms, 30-100 ms, 40-110 ms, 50-120 ms, 60-130 ms, 70-140 ms, 80-150 ms, 90-160 ms, 100-170 ms, 110-180 ms, 120-190 ms, 130-200 ms, 10-90 ms, 20-100 ms, 30-110 ms, 40-120 ms, 50-130 ms, 60-140 ms, 70-150 ms, 80-160 ms, 90-170 ms, 100-180 ms, 110-190 ms, 120-200 ms, 10-100 ms, 20-110 ms, 30-120 ms, 40-130 ms, 50-140 ms, 60-150 ms, 70-160 ms, 80-170 ms, 90-180 ms, 100-190 ms, 110-200 ms, 10-110 ms, 20-120 ms, 30-130 ms, 40-140 ms, 50-150 ms, 60-160 ms, 70-170 ms, 80-180 ms, 90-190 ms, 100-200 ms, 10-120 ms, 20-130 ms, 30-140 ms, 40-150 ms, 50-160 ms, 60-170 ms, 70-180 ms, 80-190 ms, 90-200 ms, 10-130 ms, 20-140 ms, 30-150 ms, 40-160 ms, 50-170 ms, 60-180 ms, 70-190 ms, 80-200 ms, 10-140 ms, 20-150 ms, 30-160 ms, 40-170 ms, 50-180 ms, 60-190 ms, 70-200 ms, 10-150 ms, 20-160 ms, 30-170 ms, 40-180 ms, 50-190 ms, 60-200 ms, 10-160 ms, 20-170 ms, 30-180 ms, 40-190 ms, 50-200 ms, 10-170 ms, 20-180 ms, 30-190 ms, 40-200 ms, 10-180 ms, 20-190 ms, 30-200 ms, 10-190 ms, and 20-200 ms. In certain aspects, the pulse duration is about 3 ms or about 100 ms.

In one implementation, focused ultrasound treatment is provided with an acoustic pressure between 0.1 MPa and 2.5 MPa. In one example, focused ultrasound treatment is provided with an acoustic pressure between 1 MPa and 2.5 MPa. In another implementation, focused ultrasound treatment is provided with a pulse duration between any of 1-1.1 MPa, 1.1-1.2 MPa, 1.2-1.3 MPa, 1.3-1.4 MPa, 1.4-1.5 MPa, 1.5-1.6 MPa, 1.6-1.7 MPa, 1.7-1.8 MPa, 1.8-1.9 MPa, 1.9-2 MPa, 2-2.1 MPa, 2.1-2.2 MPa, 2.2-2.3 MPa, 2.3-2.4 MPa, 2.4-2.5 MPa, 1-1.2 MPa, 1.1-1.3 MPa, 1.2-1.4 MPa, 1.3-1.5 MPa, 1.4-1.6 MPa, 1.5-1.7 MPa, 1.6-1.8 MPa, 1.7-1.9 MPa, 1.8-2 MPa, 1.9-2.1 MPa, 2-2.2 MPa, 2.1-2.3 MPa, 2.2-2.4 MPa, 2.3-2.5 MPa, 1-1.3 MPa, 1.1-1.4 MPa, 1.2-1.5 MPa, 1.3-1.6 MPa, 1.4-1.7 MPa, 1.5-1.8 MPa, 1.6-1.9 MPa, 1.7-2 MPa, 1.8-2.1 MPa, 1.9-2.2 MPa, 2-2.3 MPa, 2, 1-2.4 MPa, 2.2-2.5 MPa, 1-1.4 MPa, 1.1-1.5 MPa, 1.2-1.6 MPa, 1.3-1.7 MPa, 1.4-1.8 MPa, 1.5-1.9 MPa, 1.6-2 MPa, 1.7-2.1 MPa, 1.8-2.2 MPa, 1.9-2.3 MPa, 2-2.4 MPa, 2.1-2.5 MPa, 1-1.5 MPa, 1.1-1.6 MPa, 1.2-1.7 MPa, 1.3-1.8 MPa, 1.4-1.9 MPa, 1.5-2 MPa, 1.6-2.1 MPa, 1.7-2.2 MPa, 1.8-2.3 MPa, 1.9-2.4 MPa, 2-2.5 MPa, 1-1.6 MPa, 1.1-1.7 MPa, 1.2-1.8 MPa, 1.3-1.9 MPa, 1.4-2 MPa, 1.5-2.1 MPa, 1.6-2.2 MPa, 1.7-2.3 MPa, 1.8-2.4 MPa, 1.9-2.5 MPa, 1-1.7 MPa, 1.1-1.8 MPa, 1.2-1.9 MPa, 1.3-2 MPa, 1.4-2.1 MPa, 1.5-2.2 MPa, 1.6-2.3 MPa, 1.7-2.4 MPa, 1.8-2.5 MPa, 1-1.8 MPa, 1.1-1.9 MPa, 1.2-2 MPa, 1.3-2.1 MPa, 1.4-2.2 MPa, 1.5-2.3 MPa, 1.6-2.4 MPa, 1.7-2.5 MPa, 1-1.9 MPa, 1.1-2 MPa, 1.2-2.1 MPa, 1.3-2.2 MPa, 1.4-2.3 MPa, 1.5-2.4 MPa, 1.6-2.5 MPa, 1-2 MPa, 1.1-2.1 MPa, 1.2-2.2 MPa, 1.3-2.3 MPa, 1.4-2.4 MPa, 1.5-2.5 MPa, 1-2.1 MPa, 1.1-2.2 MPa, 1.2-2.3 MPa, 1.3-2.4 MPa, 1.4-2.5 MPa, 1-2.2 MPa, 1.1-2.3 MPa, 1.2-2.4 MPa, 1.3-2.5 MPa, 1-2.3 MPa, 1.1-2.4 MPa, 1.2-2.5 MPa, 1-2.4 MPa, 1.1-2.5 MPa, and 1-2.5 MPa. In certain aspects, the acoustic pressure is greater than zero but less than about 2 MPa, such as about 0.55 MPa or about 1.9 MPa.

In one implementation, a session of focused ultrasound treatment lasts between three minutes and seven minutes. In another implementation, a session of focused ultrasound treatment lasts between five minutes and twenty minutes. In a further implementation, a session of focused ultrasound treatment lasts between three minutes and ten minutes. In a further implementation, a session of focused ultrasound treatment lasts between five minutes and twenty minutes. In a further implementation, a session of focused ultrasound treatment lasts between ten minutes and twenty minutes. In a further implementation, a session of focused ultrasound treatment lasts between ten minutes and thirty minutes. In a further implementation, a session of focused ultrasound treatment lasts between ten minutes and sixty minutes.

In one implementation, focused ultrasound treatment is provided with a carrier frequency between 0.22 MHz and 3 MHz. In another implementation, focused ultrasound treatment is provided with a frequency having a range that is one of the following: 0.02-0.1 MHz, 0.1-0.2 MHz, 0.2-0.3 MHz, 0.3-0.4 MHz, 0.4-0.5 MHz, 0.5-0.6 MHz, 0.6-0.7 MHz, 0.7-0.8 MHz, 0.8-0.9 MHz, 0.9-1 MHz, 1-1.1 MHz, 1.1-1.2 MHz, 1.2-1.3 MHz, 1.3-1.4 MHz, 1.4-1.5 MHz, 1.5-1.6 MHz, 1.6-1.7 MHz, 1.7-1.8 MHz, 1.8-1.9 MHz, 1.9-2 MHZ, 2-2.1 MHz, 2.1-2.2 MHz, 2.2-2.3 MHz, 2.3-2.4 MHz, 2.4-2.5 MHz, 2.5-2.6 MHz, 2.6-2.7 MHz, 2.7-2.8 MHZ, 2.8-2.9 MHz, 2.9-3 MHz, 0.02-0.2 MHz, 0.1-0.3 MHz, 0.2-0.4 MHz, 0.3-0.5 MHz, 0.4-0.6 MHz, 0.5-0.7 MHz, 0.6-0.8 MHz, 0.7-0.9 MHz, 0.8-1 MHz, 0.9-1.1 MHz, 1-1.2 MHz, 1.1-1.3 MHz, 1.2-1.4 MHZ, 1.3-1.5 MHz, 1.4-1.6 MHz, 1.5-1.7 MHz, 1.6-1.8 MHz, 1.7-1.9 MHz, 1.8-2 MHz, 1.9-2.1 MHZ, 2-2.2 MHZ, 2.1-2.3 MHZ, 2.2-2.4 MHZ, 2.3-2.5 MHz, 2.4-2.6 MHz, 2.5-2.7 MHz, 2.6-2.8 MHz, 2.7-2.9 MHz, 2.8-3 MHz, 0.02-0.3 MHz, 0.1-0.4 MHz, 0.2-0.5 MHz, 0.3-0.6 MHz, 0.4-0.7 MHz, 0.5-0.8 MHz, 0.6-0.9 MHz, 0.7-1 MHz, 0.8-1.1 MHz, 0.9-1.2 MHz, 1-1.3 MHz, 1.1-1.4 MHZ, 1.2-1.5 MHz, 1.3-1.6 MHz, 1.4-1.7 MHz, 1.5-1.8 MHz, 1.6-1.9 MHz, 1.7-2 MHZ, 1.8-2.1 MHZ, 1.9-2.2 MHZ, 2-2.3 MHz, 2.1-2.4 MHz, 2.2-2.5 MHz, 2.3-2.6 MHz, 2.4-2.7 MHz, 2.5-2.8 MHZ, 2.6-2.9 MHz, 2.7-3 MHz, 0.02-0.4 MHz, 0.1-0.5 MHz, 0.2-0.6 MHz, 0.3-0.7 MHz, 0.4-0.8 MHz, 0.5-0.9 MHz, 0.6-1 MHz, 0.7-1.1 MHZ, 0.8-1.2 MHZ, 0.9-1.3 MHz, 1-1.4 MHz, 1.1-1.5 MHz, 1.2-1.6 MHz, 1.3-1.7 MHz, 1.4-1.8 MHz, 1.5-1.9 MHZ, 1.6-2 MHz, 1.7-2.1 MHz, 1.8-2.2 MHZ, 1.9-2.3 MHZ, 2-2.4 MHz, 2.1-2.5 MHz, 2.2-2.6 MHz, 2.3-2.7 MHz, 2.4-2.8 MHz, 2.5-2.9 MHz, 2.6-3 MHz, 0.02-0.5 MHz, 0.1-0.6 MHz, 0.2-0.7 MHz, 0.3-0.8 MHz, 0.4-0.9 MHz, 0.5-1 MHZ, 0.6-1.1 MHz, 0.7-1.2 MHz, 0.8-1.3 MHz, 0.9-1.4 MHz, 1-1.5 MHz, 1.1-1.6 MHz, 1.2-1.7 MHz, 1.3-1.8 MHz, 1.4-1.9 MHz, 1.5-2 MHZ, 1.6-2.1 MHz, 1.7-2.2 MHZ, 1.8-2.3 MHz, 1.9-2.4 MHZ, 2-2.5 MHz, 2.1-2.6 MHz, 2.2-2.7 MHz, 2.3-2.8 MHz, 2.4-2.9 MHz, 2.5-3 MHz, 0.02-0.6 MHZ, 0.1-0.7 MHz, 0.2-0.8 MHz, 0.3-0.9 MHz, 0.4-1 MHz, 0.5-1.1 MHz, 0.6-1.2 MHz, 0.7-1.3 MHZ, 0.8-1.4 MHZ, 0.9-1.5 MHz, 1-1.6 MHz, 1.1-1.7 MHz, 1.2-1.8 MHz, 1.3-1.9 MHz, 1.4-2 MHz, 1.5-2.1 MHz, 1.6-2.2 MHz, 1.7-2.3 MHz, 1.8-2.4 MHz, 1.9-2.5 MHz, 2-2.6 MHz, 2.1-2.7 MHz, 2.2-2.8 MHz, 2.3-2.9 MHz, 2.4-3 MHz, 0.02-0.7 MHz, 0.1-0.8 MHz, 0.2-0.9 MHz, 0.3-1 MHZ, 0.4-1.1 MHz, 0.5-1.2 MHz, 0.6-1.3 MHz, 0.7-1.4 MHZ, 0.8-1.5 MHz, 0.9-1.6 MHz, 1-1.7 MHz, 1.1-1.8 MHz, 1.2-1.9 MHz, 1.3-2 MHz, 1.4-2.1 MHz, 1.5-2.2 MHZ, 1.6-2.3 MHz, 1.7-2.4 MHZ, 1.8-2.5 MHz, 1.9-2.6 MHz, 2-2.7 MHz, 2.1-2.8 MHz, 2.2-2.9 MHZ, 2.3-3 MHZ, 0.02-0.8 MHZ, 0.1-0.9 MHz, 0.2-1 MHz, 0.3-1.1 MHz, 0.4-1.2 MHz, 0.5-1.3 MHz, 0.6-1.4 MHz, 0.7-1.5 MHZ, 0.8-1.6 MHz, 0.9-1.7 MHz, 1-1.8 MHz, 1.1-1.9 MHz, 1.2-2 MHz, 1.3-2.1 MHz, 1.4-2.2 MHZ, 1.5-2.3 MHz, 1.6-2.4 MHZ, 1.7-2.5 MHz, 1.8-2.6 MHz, 1.9-2.7 MHz, 2-2.8 MHz, 2.1-2.9 MHZ, 2.2-3 MHz, 0.02-0.9 MHZ, 0.1-1 MHz, 0.2-1.1 MHz, 0.3-1.2 MHz, 0.4-1.3 MHz, 0.5-1.4 MHZ, 0.6-1.5 MHz, 0.7-1.6 MHz, 0.8-1.7 MHz, 0.9-1.8 MHz, 1-1.9 MHz, 1.1-2 MHz, 1.2-2.1 MHZ, 1.3-2.2 MHz, 1.4-2.3 MHz, 1.5-2.4 MHZ, 1.6-2.5 MHz, 1.7-2.6 MHz, 1.8-2.7 MHz, 1.9-2.8 MHz, 2-2.9 MHZ, 2.1-3 MHz, 0.02-1 MHz, 0.1-1.1 MHz, 0.2-1.2 MHz, 0.3-1.3 MHz, 0.4-1.4 MHz, 0.5-1.5 MHz, 0.6-1.6 MHz, 0.7-1.7 MHz, 0.8-1.8 MHz, 0.9-1.9 MHz, 1-2 MHz, 1.1-2.1 MHz, 1.2-2.2 MHz, 1.3-2.3 MHz, 1.4-2.4 MHz, 1.5-2.5 MHz, 1.6-2.6 MHz, 1.7-2.7 MHz, 1.8-2.8 MHz, 1.9-2.9 MHz, 2-3 MHz, 0.02-1.5 MHz, 1.5-3 MHz. 0.02-2 MHz, and 1-3 MHz. In certain aspects, the carrier frequency is between about 0.22 and about 0.65 MHz. In certain aspects, the carrier frequency is about 0.6 MHz.

In one implementation, focused ultrasound treatment is provided with a spatial-peak temporal average intensity between 0.1 W/cm² and 8.5 W/cm². In another implementation, focused ultrasound treatment is provided with a spatial peak average intensity is between 3 W/cm² and 8.5 W/cm². In a further embodiment, focused ultrasound treatment is provided with a spatial-peak pulse-average intensity having a range that is one of the following: 0.1-0.3 W/cm², 0.2-0.4 W/cm², 0.3-0.5 W/cm², 0.4-0.6 W/cm², 0.5-0.7 W/cm², 0.6-0.8 W/cm², 0.7-0.9 W/cm², 0.8-1 W/cm², 0.9-1.1 W/cm², 1-1.2 W/cm², 1.1-1.3 W/cm², 1.2-1.4 W/cm², 1.3-1.5 W/cm², 1.4-1.6 W/cm², 1.5-1.7 W/cm², 1.6-1.8 W/cm², 1.7-1.9 W/cm², 1.8-2 W/cm², 1.9-2.1 W/cm², 2-2.2 W/cm², 2.1-2.3 W/cm², 2.2-2.4 W/cm², 2.3-2.5 W/cm², 2.4-2.6 W/cm², 2.5-2.7 W/cm², 2.6-2.8 W/cm², 2.7-2.9 W/cm², 2.8-3 W/cm², 2.9-3.1 W/cm², 3-3.2 W/cm², 3.1-3.3 W/cm², 3.2-3.4 W/cm², 3.3-3.5 W/cm², 3.4-3.6 W/cm², 3.5-3.7 W/cm², 3.6-3.8 W/cm², 3.7-3.9 W/cm², 3.8-4 W/cm², 3.9-4.1 W/cm², 4-4.2 W/cm², 4.1-4.3 W/cm², 4.2-4.4 W/cm², 4.3-4.5 W/cm², 4.4-4.6 W/cm², 4.5-4.7 W/cm², 4.6-4.8 W/cm², 4.7-4.9 W/cm², 4.8-5 W/cm², 0.1-0.6 W/cm², 0.2-0.7 W/cm², 0.3-0.8 W/cm², 0.4-0.9 W/cm², 0.5-1 W/cm², 0.6-1.1 W/cm², 0.7-1.2 W/cm², 0.8-1.3 W/cm², 0.9-1.4 W/cm², 1-1.5 W/cm², 1.1-1.6 W/cm², 1.2-1.7 W/cm², 1.3-1.8 W/cm², 1.4-1.9 W/cm², 1.5-2 W/cm², 1.6-2.1 W/cm², 1.7-2.2 W/cm², 1.8-2.3 W/cm², 1.9-2.4 W/cm², 2-2.5 W/cm², 2.1-2.6 W/cm², 2.2-2.7 W/cm², 2.3-2.8 W/cm², 2.4-2.9 W/cm², 2.5-3 W/cm², 2.6-3.1 W/cm², 2.7-3.2 W/cm², 2.8-3.3 W/cm², 2.9-3.4 W/cm², 3-3.5 W/cm², 3.1-3.6 W/cm², 3.2-3.7 W/cm², 3.3-3.8 W/cm², 3.4-3.9 W/cm², 3.5-4 W/cm², 3.6-4.1 W/cm², 3.7-4.2 W/cm², 3.8-4.3 W/cm², 3.9-4.4 W/cm², 4-4.5 W/cm², 4.1-4.6 W/cm², 4.2-4.7 W/cm², 4.3-4.8 W/cm², 4.4-4.9 W/cm², 4.5-5 W/cm², 0.1-1 W/cm², 0.2-1.1 W/cm², 0.3-1.2 W/cm², 0.4-1.3 W/cm², 0.5-1.4 W/cm², 0.6-1.5 W/cm², 0.7-1.6 W/cm², 0.8-1.7 W/cm², 0.9-1.8 W/cm², 1-1.9 W/cm², 1.1-2 W/cm², 1.2-2.1 W/cm², 1.3-2.2 W/cm², 1.4-2.3 W/cm², 1.5-2.4 W/cm², 1.6-2.5 W/cm², 1.7-2.6 W/cm², 1.8-2.7 W/cm², 1.9-2.8 W/cm², 2-2.9 W/cm², 2.1-3 W/cm², 2.2-3.1 W/cm², 2.3-3.2 W/cm², 2.4-3.3 W/cm², 2.5-3.4 W/cm², 2.6-3.5 W/cm², 2.7-3.6 W/cm², 2.8-3.7 W/cm², 2.9-3.8 W/cm², 3-3.9 W/cm², 3.1-4 W/cm², 3.2-4.1 W/cm², 3.3-4.2 W/cm², 3.4-4.3 W/cm², 3.5-4.4 W/cm², 3.6-4.5 W/cm², 3.7-4.6 W/cm², 3.8-4.7 W/cm², 3.9-4.8 W/cm², 4-4.9 W/cm², 4.1-5 W/cm², 0.1-2 W/cm², 0.2-2.1 W/cm², 0.3-2.2 W/cm², 0.4-2.3 W/cm², 0.5-2.4 W/cm², 0.6-2.5 W/cm², 0.7-2.6 W/cm², 0.8-2.7 W/cm², 0.9-2.8 W/cm², 1-2.9 W/cm², 1.1-3 W/cm², 1.2-3.1 W/cm², 1.3-3.2 W/cm², 1.4-3.3 W/cm², 1.5-3.4 W/cm², 1.6-3.5 W/cm², 1.7-3.6 W/cm², 1.8-3.7 W/cm², 1.9-3.8 W/cm², 2-3.9 W/cm², 2.1-4 W/cm², 2.2-4.1 W/cm², 2.3-4.2 W/cm², 2.4-4.3 W/cm², 2.5-4.4 W/cm², 2.6-4.5 W/cm², 2.7-4.6 W/cm², 2.8-4.7 W/cm², 2.9-4.8 W/cm², 3-4.9 W/cm², 3.1-5 W/cm², 0.1-3 W/cm², 0.2-3.1 W/cm², 0.3-3.2 W/cm², 0.4-3.3 W/cm², 0.5-3.4 W/cm², 0.6-3.5 W/cm², 0.7-3.6 W/cm², 0.8-3.7 W/cm², 0.9-3.8 W/cm², 1-3.9 W/cm², 1.1-4 W/cm², 1.2-4.1 W/cm², 1.3-4.2 W/cm², 1.4-4.3 W/cm², 1.5-4.4 W/cm², 1.6-4.5 W/cm², 1.7-4.6 W/cm², 1.8-4.7 W/cm², 1.9-4.8 W/cm², 2-4.9 W/cm², 2.1-5 W/cm², 0.1-4 W/cm², 0.2-4.1 W/cm², 0.3-4.2 W/cm², 0.4-4.3 W/cm², 0.5-4.4 W/cm², 0.6-4.5 W/cm², 0.7-4.6 W/cm², 0.8-4.7 W/cm², 0.9-4.8 W/cm², 1-4.9 W/cm², 1.1-5 W/cm², 5-5.2 W/cm², 5.1-5.3 W/cm², 5.2-5.4 W/cm², 5.3-5.5 W/cm², 5.4-5.6 W/cm², 5.5-5.7 W/cm², 5.6-5.8 W/cm², 5.7-5.9 W/cm², 5.8-6 W/cm², 5.9-6.1 W/cm², 6-6.2 W/cm², 6.1-6.3 W/cm², 6.2-6.4 W/cm², 6.3-6.5 W/cm², 6.4-6.6 W/cm², 6.5-6.7 W/cm², 6.6-6.8 W/cm², 6.7-6.9 W/cm², 6.8-7 W/cm², 6.9-7.1 W/cm², 6-7.2 W/cm², 7.1-7.3 W/cm², 7.2-7.4 W/cm², 7.3-7.5 W/cm², 7.4-7.6 W/cm², 7.5-7.7 W/cm², 7.6-7.8 W/cm², 7.7-7.9 W/cm², 7.8-8 W/cm², 7.9-8.1 W/cm², 8-8.2 W/cm², 8.1-8.3 W/cm², 8.2-8.4 W/cm², 8.3-8.5 W/cm², 5-5.5 W/cm², 5.1-5.6 W/cm², 5.2-5.7 W/cm², 5.3-5.8 W/cm², 5.4-5.9 W/cm², 5.5-6 W/cm², 5.6-6.1 W/cm², 5.7-6.2 W/cm², 5.8-6.3 W/cm², 5.9-6.4 W/cm², 6-6.5 W/cm², 6.1-6.6 W/cm², 6.2-6.7 W/cm², 6.3-6.8 W/cm², 6.4-6.9 W/cm², 6.5-7 W/cm², 6.6-7.1 W/cm², 6.7-7.2 W/cm², 6.8-7.3 W/cm², 6.9-7.4 W/cm², 7-7.5 W/cm², 7.1-7.6 W/cm², 7.2-7.7 W/cm², 7.3-7.8 W/cm², 7.4-7.9 W/cm², 7.5-8 W/cm², 7.6-8.1 W/cm², 7.7-8.2 W/cm², 7.8-8.3 W/cm², 7.9-8.4 W/cm², 8-8.5 W/cm², 5.1-6 W/cm², 5.2-6.1 W/cm², 5.3-6.2 W/cm², 5.4-6.3 W/cm², 5.5-6.4 W/cm², 5.6-6.5 W/cm², 5.7-6.6 W/cm², 5.8-6.7 W/cm², 5.9-6.8 W/cm², 6-6.9 W/cm², 6.1-7 W/cm², 6.2-7.1 W/cm², 6.3-7.2/cm², 6.4-7.3 W/cm², 6.5-7.4 W/cm², 6.6-7.5 W/cm², 6.7-7.6 W/cm², 6.8-7.7 W/cm², 6.9-7.8 W/cm², 7-7.9 W/cm², 7.1-8 W/cm², 7.2-8.1 W/cm², 7.3-8.2/cm², 7.4-8.3 W/cm², 7.5-8.4 W/cm², 7.6-8.5 W/cm², 5.1-7 W/cm², 5.2-7.1 W/cm², 5.3-7.2 W/cm², 5.4-7.3 W/cm², 5.5-7.4 W/cm², 5.6-7.5 W/cm², 5.7-7.6 W/cm², 5.8-7.7 W/cm², 5.9-7.8 W/cm², 6-7.9 W/cm², 6.1-8 W/cm², 6.2-8.1 W/cm², 6.3-8.2 W/cm², 6.4-8.3 W/cm², 6.5-8.4 W/cm², 6.6-8.5 W/cm², and 5.1-8.5 W/cm². In certain aspects, the spatial-peak temporal average intensity is about 7.33 W/cm²

In certain aspects, the I_{SPPA} can greater than zero and up to about 110 W/cm². In certain aspects, the I_{SPTA} can be greater than zero and up to about 8 W/cm². In certain aspects, the duty cycle can be greater than 0% and less about than 30%. In certain aspects, the duty cycle can be about 6.6%. In certain aspects, the pulse repetition rate can be between about 0.33 Hz and about 100 Hz. In certain aspects, the pulse repetition rate is about 0.33 Hz. In certain aspects, the pulse duration is between about 3 ms and about 200 ms. In certain aspects, the pulse duration is about 100 ms. In certain aspects, the acoustic pressure is between about 0.1 MPa and about 2.5 MPa. In certain aspects, the acoustic pressure is about 2 MPa. In certain aspects, the mechanical index is between about 0.1 and about 5. In certain aspects, the mechanical index is 4. In certain aspects, a session of focused ultrasound treatment lasts greater than zero minutes and up to about 30 minutes.

### Medical Conditions

The present disclosure provides methods of improving a medical condition in a patient in need thereof. The methods include delivering a focused ultrasound signal to a neural target site of the patient's ANS. The medical condition can comprise, for example, chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), post-traumatic stress disorder (PTSD), anxiety and anxiety-associated disorders, autism, fibromyalgia, uterine function, metabolic disorder, urinary incontinence, or binge eating.

Also provided herein are computer programs comprising instructions, which, when the program is executed by a processor for controlling an ultrasound device or transducer causes the steps of delivering a focused ultrasound signal to a neural site of the ANS. The ultrasound signal can delivered to a neural target site of the ANS sufficient to improve, for example, chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence, or binge eating.

### Target Sites

The ANS neural target site can be a site of the sympathetic nervous system (SNS) and/or the parasympathetic nervous system (PSNS). Such sites include autonomic nerves (including pre- and post-ganglionic fibers of the ANS), autonomic ganglia, and autonomic plexus. Target sites of the SNS include a sympathetic ganglion, a sympathetic nerve or a sympathetic plexus. Regarding sympathetic ganglia, the neural target site can be a prevertebral ganglion or a paravertebral ganglion (sympathetic nerve chain ganglion) or the sympathetic trunk. Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus.

### Systems

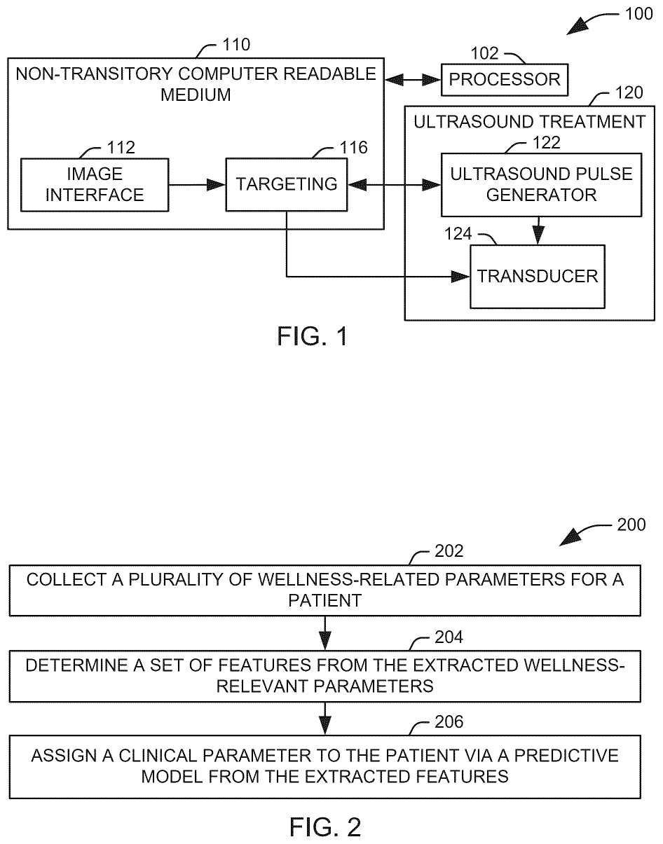

FIG. 1 illustrates a system 100 for providing focused ultrasound treatment for treatment or diagnosis of a disorder to a specific region of interest. The system 100 includes a processor 102 and a non-transitory computer readable medium 110 that stores executable instructions for targeting focused ultrasound treatment for treatment or diagnosis of disorders. The executable instructions include an imaging interface 112 that receives an image representing a region of interest for a patient from one or more associated imaging systems (not shown). The imaging interface 112 can include appropriate software components for communicating with an imaging system (not shown) or repository of stored images (not shown) over a network via a network interface (not shown) or via a bus connection. The image can be provided to a targeting component 116 that selects a location and intensity profile for the focused ultrasound treatment based on the image, the location, the type of tissue, and one or more feedback parameters of the patient, such as those listed in Tables II through VIII below.

In one example, the targeting component 116 can operate in conjunction with a focused ultrasound treatment system 120, comprising an ultrasound pulse generator 122 and at least one transducer 124 driven by the ultrasound pulse generator. Specifically, the targeting component can instruct the ultrasound pulse generator 122 to deliver a focused ultrasound neuromodulation signal to a neural site of the autonomic nervous system, with the ultrasound signal having a mechanical index of between about 1 and about 4 and/or an acoustic pressure of between about 1 MPa and about 2.5 MPa to a site of the ANS sufficient to improve chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), post-traumatic stress disorder (PTSD), anxiety and anxiety-associated disorders, autism, fibromyalgia, uterine function, metabolic disorder, urinary incontinence, or binge eating.

### Methods of Screening

FIG. 2 represents a method 200 for screening a patient for a specific disorder. At 202, a plurality of parameters is collected for the patient. Parameters can be retrieved from an electronic health records (EHR) interface and/or other available databases, including, for example, employment information (e.g., title, department, shift), age, sex, home zip code, genomic data, nutritional information, medication intake, household information (e.g., type of home, number and age of residents), social and psychosocial data, consumer spending and profiles, financial data, food safety information, the presence or absence of physical abuse, and relevant medical history. Parameters can also be measured via diagnostic systems, imaging systems, wearable sensors, cognitive tests, questionnaires, and other means. Relevant parameters can include at least physiological, cognitive, motor/musculoskeletal, sensory, sleep, biomarkers and behavioral parameters.

In aspects where a patient's physiological parameter is measured, the physiological parameter can be a response of the patient's autonomic nervous system to cue exposure and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, brain activity, electrodermal activity, body temperature, and blood oxygen saturation level. Table II provides non-limiting examples of physiological parameters that can be measured and exemplary tests to measure the physiological parameters.

### Clinical/Feedback Parameters

TABLE II

| Physiological | Exemplary Devices and Methods to Measure |
|---|---|
| Parameter | Physiological Parameters |
| Brain Activity & | Electroencephalogram, Magnetic Resonance |
| Brain Structure | Imaging (MRI), including functional |
| | Magnetic Resonance Imaging (fMRI), |
| | positron emission tomography (PET), |
| | single-photon emission computer tomography |
| | (SPECT), magnetoencephalography (MEG), |
| | near-infrared spectroscopy (NIRS), functional |
| | near-infrared spectroscopy (f-NIRS), |
| | transcranial doppler ultrasound (TCD), and |
| | other brain imaging modalities looking at |
| | electrical, blood flow, neurotransmitter, and |
| | metabolic MRI, taken either during a cognitive |
| | task or while the patient is at rest |
| Heart rate | Electrocardiogram and Photoplethysmogram |
| Heart rate | Electrocardiogram, Photoplethysmogram |
| variability | |
| Eye tracking | Pupillometry, including tracking saccades, |
| | fixations, and pupil size (e.g., dilation) |
| Perspiration | Perspiration sensor |
| Blood pressure | Sphygmomanometer |
| Body temperature | Thermometer, infrared thermography |
| Blood oxygen | Pulse oximeter/accelerometer |
| saturation and | |
| respiratory rate | |
| Skin conductivity | Electrodermal activity |
| Facial emotions | Camera or EMG based sensors for |
| | emotion and wellness |
| Sympathetic and | Derived from the above measurements |
| parasympathetic | |
| tone | |

The physiological parameters can be measured in clinical settings with appropriate devices or in non-clinical settings via wearable, implantable, or portable devices. Some information can also be determined from self-reporting by the user via applications in a mobile device or via interaction with applications on the mobile device. For example, a smart watch, ring, or patch can be used to measure the user's heart rate, heart rate variability, body temperature, blood oxygen saturation, movement, and sleep. In a non-clinical setting, these values can also be subject to a diurnal analysis to estimate variability. Eye tracking can be performed, for example, using a camera on a mobile device and specialized software.

Table III provides non-limiting examples of cognitive parameters that are gamified and that can be measured and exemplary methods and tests/tasks to measure such cognitive parameters. The cognitive parameters can be assessed by a battery of cognitive tests that measure, for example, executive function, decision making, working memory, attention, and fatigue.

TABLE III

| | Exemplary Tests and Methods to Measure |
|---|---|
| Cognitive Parameter | Cognitive Parameters |
| Temporal discounting | Kirby Delay Discounting Task |
| Alertness and fatigue | Psychomotor Vigilance Task, go/no-go, |
| | reaction time, |
| Focused attention and | Erikson Flanker Task |
| response inhibition | |
| Working memory | N-Back Task, Change detection, spatial |
| | verbal and visual working memory tests |
| Attentional bias towards | Dot-Probe Task |
| emotional cues | |
| Inflexible persistence | Wisconsin Card Sorting Task |
| Decision making | Iowa Gambling Task |
| Risk taking behavior | Balloon Analogue Risk Task |
| Inhibitory control | Saccade and Anti-Saccade Task |
| Sustained attention | Sustained Attention |
| Executive function | Task Shifting or Set Shifting Task |
| Long term memory | Identifying pictures of famous people and |
| | other memory related tasks |

These cognitive tests can be administered in a clinical/laboratory setting or in a naturalistic, non-clinical setting such as when the user is at home, work or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch. In one example, the patient can be allowed to explore a virtual reality environment and collect items within the environment. The patient is then asked to recount where each item was found within the virtual environment and the relationship of that location to a starting point, testing the patient's ability to recall spatial relationships among the virtual locations.

TABLE IV provides non-limiting examples of parameters associated with movement and activity of the user, referred to herein alternatively for ease of reference as “motor parameters,” that can be measured and exemplary tests, devices, and methods. The use of portable monitoring, physiological sensing, and portable computing devices allows the motor parameters to be measured. Using embedded accelerometer, GPS, and cameras, the user's movements can be captured and quantified to see how wellness affects them and related to the parameters. Range of motion and gait analysis can be performed in a clinical setting using appropriate motion capture and camera equipment for evaluation.

TABLE IV

| Motor/Mus- | |
|---|---|
| culoskeletal | Exemplary Tests and Methods to Measure |
| Parameter | Motor/Musculoskeletal Parameters |
| Activity | Daily movement total, time of activities, |
| level | from wearable accelerometer, steps, |
| | Motion Capture data, gait analysis, GPS, |
| | deviation from established geolocation |
| | patterns, force plates |
| Gait analysis | Gait mat, camera, force plates |
| Range of | Motion capture, camera, |
| motion | |
| Motor | Time up and go, 6-min walk test, |
| | perceptual motor tasks |

TABLE V provides non-limiting examples of parameters associated with sensory acuity of the user, referred to herein alternatively for ease of reference as “sensory parameters,” that can be measured and exemplary tests, devices, and methods.

TABLE V

| Sensory | Exemplary Tests and Methods to Measure |
|---|---|
| Parameter | sensory Parameters |
| Vision | Visual acuity test, visual field tests, eye |
| | tracking, EMG |
| Hearing | Hearing tests |
| Touch | Two-point discrimination, frey filament |
| Smell/taste | |
| Vestibular | Vestibula function test |

TABLE VI provides non-limiting examples of parameters associated with a sleep quantity, phases, and quality of the user, referred to herein alternatively for ease of reference as “sleep parameters,” that can be measured and exemplary tests, devices, and methods.

TABLE VI

| Sleep | Exemplary Tests and Methods to Measure |
|---|---|
| Parameter | Sleep Parameters |
| Sleep from | Sleep onset & offset, sleep quality, sleep |
| wearables | quantity, from wearable accelerometer, |
| | temperature, and PPG, |
| Sleep | Pittsburg Sleep Quality Index, Functional |
| Questions | Outcomes of Sleep Questionnaire, Fatigue |
| | Severity Scale, Epworth Sleepiness Scale |
| Devices | Polysomnography; ultrasound, camera, |
| | bed sensors, EEG |
| Circadian | Light sensors, actigraphy, serum levels, |
| Rhythm | core body temperature |

TABLE VII provides non-limiting examples of parameters extracted by locating biomarkers associates with the user, referred to herein alternatively for ease of reference as “biomarker parameters,” that can be measured and exemplary tests, devices, and methods. Biomarkers can also include imaging and physiological biomarkers related to a state of chronic wellness and improvement or worsening of the chronic wellness state.

TABLE VII

| | Exemplary Tests and Methods to Measure |
|---|---|
| Biomarkers Parameter | Biomarkers Parameters |
| Genetic biomarkers | Genetic testing |
| Immune biomarkers | Blood, saliva, and/or urine tests |
| including TNF-alpha, | |
| immune alteration | |
| (e.g., ILs), oxidative | |
| stress, and hormones | |
| (e.g., cortisol) | |

Table VIII provides non-limiting examples of psychosocial and behavioral parameters, referred to herein alternatively for ease of reference as “psychosocial parameters,” that can be measured and exemplary tests, devices, and methods.

TABLE VIII

| Psychosocial or | Exemplary Tests and Methods to Measure |
|---|---|
| Behavioral Parameter | Psychosocial or Behavioral Parameters |
| Symptom log | Presence of specific symptoms (i.e., fever, |
| | headache, cough, loss of smell) |
| Medical Records | Medical history, prescriptions, setting for |
| | treatment devices such as spinal cord |
| | stimulator, imaging data |
| Wellness Rating | Visual Analog Scale, Defense & Veterans |
| | wellness rating scale, wellness scale, |
| | Wellness Assessment screening tool and |
| | outcomes registry |
| Burnout | Burnout inventory or similar |
| Physical, Mental, and | User-Reported Outcomes Measurement |
| Social Health | Information System (PROMIS), Quality |
| | of Life Questionnaire |
| Depression | Hamilton Depression Rating Scale |
| Anxiety | Hamilton Anxiety Rating Scale |
| Mania | Snaith-Hamilton Pleasure Scale |
| Mood/ | Profile of Mood States; Positive Affect |
| Catastrophizing scale | Negative Affect Schedule |
| Affect | Positive Affect Negative Affect Schedule |
| Impulsivity | Barratt Impulsiveness Scale |
| Adverse Childhood | Childhood trauma |
| Experiences | |
| Daily Activities | Exposure, risk taking |
| Daily Workload and | NASA Task Load Index, Perceived Stress |
| Stress | Scale (PSS), Social Readjustment Rating |
| | Scale (SRRS) |
| Social Determents | Social determents of health questionnaire |
| of Health | |

Additional elements of monitoring can include the monitoring of the user's compliance with the use of a smart phone, TV, portable device, a portable device. For example, a user may be sent messages by the system inquiring on their wellness level, general mood, or the status of any other parameter on the portable computing device. A measure of compliance can be determined according to the percentage of these messages to which the user responds via the user interface on the portable computing device and used as an indication as to how likely the patient is to comply with treatment protocols and monitoring. Further, where parameters cannot be readily extracted from wearable or portable devices, they can be retrieved from an electronic health records (EHR) database. Biomarker and motion parameters, in particular, may be retrieved from the EHR, along with other parameters including medical history, prescribed medications, demographic parameters, age, height, weight, and other medically relevant parameters.

At 204, a set of features are determined from the extracted parameters as categorical and continuous values representing the parameters. In one example, the values can include descriptive statistics, such as measures of central tendency (e.g., median, mode, arithmetic mean, or geometric mean) and measures of deviation (e.g., range, interquartile range, variance, standard deviation, etc.) of time series of the monitored parameters, as well as the time series themselves. Specifically, the feature set provided to the predictive model can include, for at least one parameter, either two values representing the value for the parameter at different times or a single value, such as a measure of central tendency or a measure of deviation which represents values for the parameter across a plurality of times.

In one implementation, features can be generated via a wavelet transform on a time series of values for one or more parameters to provide a set of wavelet coefficients. It will be appreciated that the wavelet transform used herein is two-dimensional, such that the coefficients can be envisioned as a two-dimensional array across time and either frequency or scale. For a given time series of values, xᵢ, the wavelet coefficients, Wₐ(n), produced in a wavelet decomposition can be defined as:

$${W}_{a} ( n ) = {a}^{- 1} {\sum }_{i = 1}^{M} {x}_{i} ψ ( \frac{i - n}{a} ) \qquad Eq . 1$$

wherein Ψ is the wavelet function, M is the length of the time series, and a and n define the coefficient computation locations.

A facial expression classifier (not shown) can evaluate recorded data from a camera and/or recorded images or videos of the user's face from a smartphone or other mobile device, to assign an emotional state to the user at various times throughout the day. The extracted features can be categorical, representing the most likely emotional state of the user, or continuous, for example, as a time series of probability values for various emotional states (e.g., anxiety, discomfort, anger, etc.) as determined by the facial expression classifier. One or more image classifiers that reduce provided medical images to categorical or continuous features for use at the predictive model. It will be appreciated that each of the facial expression classifier and the one or more image classifiers can be implemented using one or more of the models discussed below for use in the predictive model.

In one implementation, the extracted features from the set of parameters can be collected into a set of aggregate parameters. It will be appreciated that each aggregate parameter can be a weighted combination of the set of parameters, functions of parameters from the set of parameters, or features extracted from the parameters. Accordingly, a given aggregate parameter can represent a plurality of parameters, and, in general, the plurality of parameters represented by each aggregate parameter will be related, such that the aggregate parameter represents a specific domain of wellness for the user. In general, each aggregate parameters can use parameters from various sources. In some implementations, the aggregate parameters can be provided to multiple predictive models (not shown) that each receive a unique proper subset of the aggregate parameters. Each predictive model can provide a different clinical parameter representing a different aspect of the user's wellness, such that the aggregate parameters can be utilized for multiple purposes in evaluating the wellness of the user. It will be appreciated that the specific parameters and features used for screening can vary with the implementation.

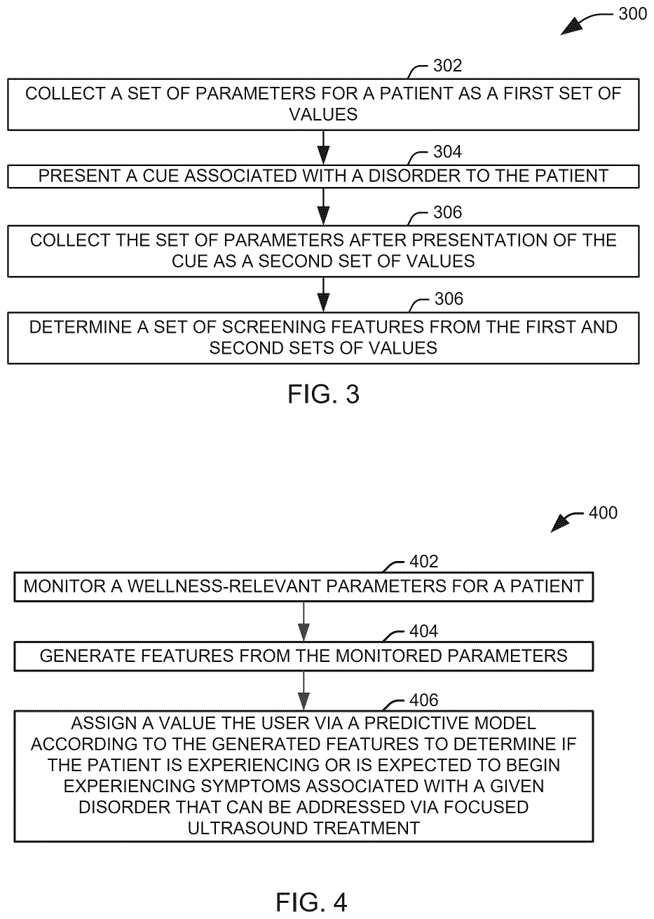

FIG. 3 illustrates a method 300 for using cues to generate features used to screen patients for a disorder. It will be appreciated that the method 300 describes eliciting responses to cues in the context of patient screening, that cue-induced responses can also be used in detecting or predicting the onset of symptoms for a patient, for determining, refining, and optimizing an appropriate target location or appropriate parameters for focused ultrasound treatment, or for evaluating the effectiveness of focused ultrasound treatment. The presentation of cues can also be used as part of treatment for various disorders, as will be discussed in more detail below. At 302, a set of parameters representing the disorder is collected from the patient as a first set of values. The specific parameters will vary with the disorder for which the patient is being screened, but in general, the parameters can include the patient's response to various standard questionnaires as well as physiological parameters measured from the patients. At 304, a cue associated with the disorder is presented to the patient. It will be appreciated that the cue will be selected to be specific to the disorder, and can include not only visual cues but auditory, gustatory, tactile, and olfactory cues. At 306, the set of parameters representing the disorder is collected from the patient after presentation of the cue as a second set of values. At 308, a set of screening features are determined from the first set of values and the second set of values. In one example, the screening features are determined as functions of the first and second values for a given parameter, such as a difference or a ratio between the values.

Returning to FIG. 2, at 206, a clinical parameter is assigned to the patient via a predictive model from the extracted features. The predictive model can utilize one or more pattern recognition algorithms, each of which analyze the extracted features or a subset of the extracted features to assign a continuous or categorical clinical parameter to the user representing the likelihood that the patient would benefit from focused ultrasound treatment. In one example, the clinical parameter can be a continuous parameter representing the likelihood that the patient has a disorder that can be treated via focused ultrasound treatment, the likelihood that the patient has a specific disorder that can be treated via focused ultrasound treatment, the likelihood that a patient will benefit from focused ultrasound treatment generally given a known diagnosis, or the likelihood that the patient will benefit from focused ultrasound treatment in a specific location. In another example, the clinical parameter can be a categorical parameter representing whether the patient has a disorder that can be treated via focused ultrasound treatment, categories representing changes in symptoms associated with a disease or disorder (e.g., “improving”, “stable, “worsening”), categories representing a predicted response to focused ultrasound treatment generally or at a specific location, whether the patient has a specific disorder that can be treated via focused ultrasound treatment, the severity of a disorder, whether a patient will benefit from focused ultrasound treatment given a known diagnosis, or categories representing ranges of likelihoods that the patient falls into one of these categories.

Where multiple classification or regression models are used, an arbitration element can be utilized to provide a coherent result from the plurality of models. The training process of a given classifier will vary with its implementation, but training generally involves a statistical aggregation of training data into one or more parameters associated with the output class. The training process can be accomplished on a remote system and/or on the local device or wearable app. The training process can be achieved in a federated or non-federated fashion. For rule-based models, such as decision trees, domain knowledge, for example, as provided by one or more human experts or extracted from existing research data, can be used in place of or to supplement training data in selecting rules for classifying a user using the extracted features. Any of a variety of techniques can be utilized for the classification algorithm, including support vector machines, regression models, self-organized maps, fuzzy logic systems, data fusion processes, boosting and bagging methods, rule-based systems, or artificial neural networks.

Federated learning (aka collaborative learning) is a predictive technique that trains an algorithm across multiple decentralized edge devices or servers holding local data samples, without exchanging their data samples. This approach stands in contrast to traditional centralized predictive techniques where all data samples are uploaded to one server, as well as to more classical decentralized approaches which assume that local data samples are identically distributed. Federated learning enables multiple actors to build a common, robust predictive model without sharing data, thus addressing critical issues such as data privacy, data security, data access rights, and access to heterogeneous data. Its applications are spread over a number of industries including defense, telecommunications, IoT, or pharmaceutics.

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. In one implementation, the SVM can be implemented via a kernel method using a linear or non-linear kernel.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier. Another example is utilizing an autoencoder to detect outlier in parameters as an anomaly detector to identify when various parameters are outside their normal range for an individual.

Many ANN classifiers are fully connected and feedforward. A convolutional neural network, however, includes convolutional layers in which nodes from a previous layer are only connected to a subset of the nodes in the convolutional layer. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. As an example, Long Short-Term Memory (LSTM) networks are a modified version of recurrent neural networks, which makes it easier to remember past data in memory.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used.

In some implementations, the predictive model can be retrained to tune various parameters of the model based upon the accuracy of predictions made by the model. Parameters associated with the model, such as internal weights and thresholds for producing categorical inputs or outputs from continuous values, can be adjusted according to the differences in the actual and predicted outcomes. In one example, an actual value for the clinical parameter for a given patient can be determined as a categorical or continuous outcome, for example, based on a degree of improvement for the patent, a diagnosis, or other appropriate outcome, and combined with the set of features for the patient to provide additional training samples for the model.

If it is determined that focused ultrasound treatment is not appropriate for the patient, the patient can be assigned to an alternative treatment. Alternative treatments can include any of medications, biologicals, surgical intervention, changes of settings for an existing stimulator device, behavioral and social intervention, digital intervention via a portable device, mindfulness approaches, social media approaches, a care provider coming to the individual, directing an individual to go to a clinic, emergency room, or hospital, or directing the user to obtain additional testing.

If it is determined that focused ultrasound treatment is appropriate, a specific focused ultrasound treatment protocol is selected for the patient. It will be appreciated that the specific focused ultrasound treatment protocol can vary with the severity of the disorder. The patient can then monitored to predict or detect an onset of symptoms. It will be appreciated that the timing for focused ultrasound treatment can be personalized and depend on both the onset of symptoms and the progression of the disorder as well as various treatment protocols associated with focused ultrasound modality and any therapeutics introduced during treatment.

FIG. 4 illustrates one method 400 for monitoring a patient to predict or detect an onset of symptoms associated with a disorder for a patient. At 402, parameters are monitored for a patient. It will be appreciated that the monitored parameters can include any of the parameters discussed in Tables II-VIII, and that the parameters can be monitored, for example, using wearable devices, portable devices, such as mobile phones, tablets, and personal computers, or from periodically querying an electronic health records system. At 404, appropriate features are generated from the monitored parameters. At least a subset of the monitored parameters can be represented by features representing a change in the parameter over time, and can be represented as a time series, or a descriptive statistic, such as a measure of central tendency or a measure of deviation which represents values for the parameter across a plurality of times.

At 406, the extracted parameters can be provided to a predictive model to determine if the patient is experiencing or is expected to begin experiences symptoms associated with a given disorder that can be addressed via focused ultrasound treatment. In one example, the predictive model can assign a continuous parameter that corresponds to a likelihood that that user has or is about to have symptoms associated with a disorder, an increase in stress for the patient, an increased in anxiety for the patient, a likelihood that the user will experience an intensifying of symptoms associated with the disorder, a current or predicted level of pain for the user, an expected performance level of the user associated with a current or future time for a particular activity or occupation, or a change in symptoms associated with a disease or disorder. In another example, the predictive model can assign a categorical parameter that corresponds to ranges of the likelihoods described above, the presence or predicted presence of a specific disease or disorder, a set of categories representing the patient's readiness for a particular activity or occupation, categories representing changes in symptoms associated with a disease or disorder (e.g., “improving”, “stable, “worsening”), or categories representing a status of the user (e.g., normal,” “stressed”, “ill”).

In one implementation, the predictive model can include a constituent model that predicts future values for the aggregate parameters, such as a convolutional neural network that is provided with one or more two-dimensional arrays of wavelet transform coefficients as an input. The wavelet coefficients detect changes not only in time, but also in temporal patterns, and can thus reflect changes in the ordinary biological rhythms of the user. It will be appreciated that a given constituent model can use data in addition to the aggregate parameters, such as other extracted features to provide these predictions. Additionally, or alternatively, the predictive model can use constituent models that predict current or future values for the aggregate parameters, with these measures then used as features for generating the output of the predictive model.

In one example, the predictive model is an anomaly detection model, which detects deviations from expected values within a feature space and determines when these deviations are significant. The anomaly detection model can be trained on data from the user, which establishes a baseline of expected values for the user, and/or on data collected from other users. In one example, training the predictive model is initially trained on data collected from other users while values for the subset of the set of parameters are collected from the user over a period of time. Once a sufficient amount of data is available for the user, the predictive model is retrained on the collected values for the subset of the set of parameters.

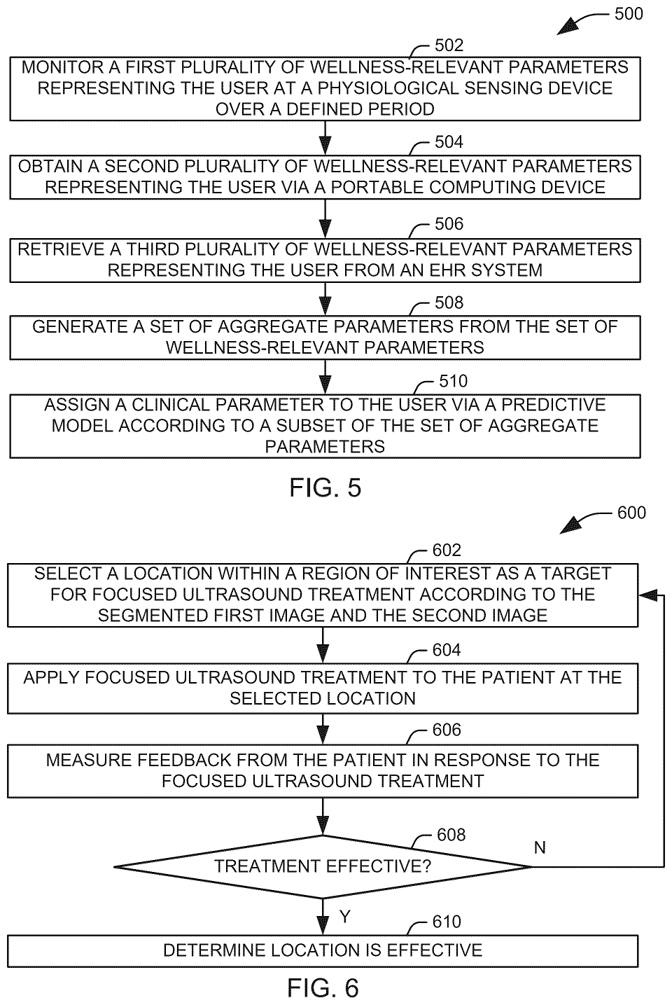

FIG. 5 illustrates another example of a method 500 for evaluating a patient for an onset of symptoms requiring treatment via focused ultrasound treatment. The result of the method is a clinical parameter representing whether the patient is experiencing or is expected to experience symptoms that would require focused ultrasound treatment. At 502, a first plurality of parameters representing the user are monitored at a physiological sensing device over a defined period. In one example, the first plurality of parameters can include parameters representing the autonomic function of the user and parameters representing the sleep and circadian rhythms of the user. At 504, a second plurality of parameters representing the user are obtained via a portable computing device. In one example, the second plurality of parameters can include parameters representing the cognitive and/or sociobehavioral wellness of the user. At 506, a third plurality of parameters representing the user are retrieved from an electronic health records (EHR) system. The third plurality of parameters can include, for example, parameters representing the musculoskeletal health, genomics, and various biomarkers of the user. The first plurality of parameters, the second plurality of parameters, and the third plurality of parameters collectively form a set of parameters.

At 508, a set of aggregate parameters are generated from the set of parameters, with each of the set of aggregate parameters comprising a unique proper subset of the set of parameters. In one example, the set of aggregate parameters includes at least a first aggregate parameter representing autonomic function of the user, a second aggregate parameter representing a cognitive function of the user, and a third aggregate parameter representing a motor and musculoskeletal health of the user. In another example, the set of aggregate parameters includes at least a first aggregate parameter representing sleep and circadian rhythms of the user, a second aggregate parameter representing a sociobehavioral function of the user, and a third aggregate parameter representing a biomarkers and genomics of the user.

At 510, a clinical parameter is assigned to the user via a predictive model according to a subset of the set of aggregate parameters. In one example, the clinical parameter is a value representing an overall wellness of the user, and the subset of the set of aggregate parameters comprises the entire set of aggregate parameters. In another example, the subset of the set of aggregate parameters is a proper subset. It will be appreciated that the aggregate parameters can be provided to multiple predictive models, with each predictive model receiving a unique subset of the set of aggregate parameters. In one example, the predictive model is an anomaly detection model, which detects deviations from expected values within a feature space and determines when these deviations are significant. The anomaly detection model can be trained on data from the user, which establishes a baseline of expected values for the user, or on data collected from other users. In one example, training the predictive model is initially trained on data collected from other users while values for the subset of the set of parameters are collected from the user over a period of time. Once a sufficient amount of data is available for the user, the predictive model is retrained on the collected values for the subset of the set of parameters.

In one example, a wavelet decomposition is performed on the time series for at least one aggregate parameter to provide a set of wavelet coefficients, and the set of wavelet coefficients or one or more values derived from the set of wavelet coefficients can be provided to the predictive model. Additionally or alternatively, the user can be assigned a predicted value representing a future value of a given aggregate parameter according to the values for the subset of aggregate parameters, and the value assigned to the user can be assigned based on the predicted value.

Additionally or alternatively, feedback, in the form of a self-reported level of symptoms, notes from a later clinical visit, or a measured future value for a parameter, can be used to refine the predictive model. For example, the self-reported or measured value can be compared to the value assigned to the user via a predictive model, and a parameter associated with the predictive model can be changed according to the comparison. In one example, this can be accomplished by generating a reward for a reinforcement learning process based on a similarity of the measured outcome to the value assigned to the user and changing the parameter via the reinforcement learning process.

It will be appreciated that each of the parameters and the value assigned to the user can be provided, for example, via a user interface or network interface, to one or more of the user, the user's health care provider, the user's care team, a research team, a user's workplace, a user's sports team, an insurer, or other interested entities. This allows the value to be used to make decisions about the user's care and activities. Feedback provided to the user can be used to improve the user's awareness, perception and interpretation of being in an overall positive and negative states, allowing the user to learn strategies for avoiding negative states and inducing positive states. The provided data can also be used for improvement or optimization of cognitive, motor, sensory, and behavioral function as well as generally attempting to improve the user's quality of life through suggesting actions for the user in response to changes in the clinical parameter. For example, a message can be transmitted to the user's portable computing device suggesting a course of action for the user when the clinical parameter is outside of a predetermined range of values.

FIG. 6 illustrates another method 600 for targeting focused ultrasound treatment in a for treatment of a disorder using acute feedback. At 602, a location within a region of interest is selected as an initial target for focused ultrasound treatment according to the segmented first image and the second image. It will be appreciated that a given treatment can be performed over a number of individual locations, and that this analysis can be performed for each of those locations. At 604, focused ultrasound treatment is applied to the selected location and feedback from the patient in response to the applied focused ultrasound treatment is measured at 606. In one implementation, administration of neuromodulation can be preceded or accompanied by priming, in which that patient is presented with a stimulus associated with their disorder to increase neural activity associated with the disorder. The feedback can include observations of a clinician on the appearance and behavior of the patient, self-reporting from the patient about symptoms of the disorder, measured electrical activity in the region of interest, and biometric parameters, such as those described in Tables II-VIII above. In particular, imaging may be used to determine the effectiveness and potential side effects at a given location. Acoustic feedback can also be used in focused ultrasound applications to evaluate both safety and effectiveness of the treatment. These imaging assessments provide quantitative personalized assessments of therapy safety, dose and benefit, and are analogous to digital fingerprinting.

At 608, an effectiveness of the focused ultrasound treatment is determined according to the measured feedback. This can be done, for example, either by a rule-based approach or by providing the measured feedback parameters to a predictive model. If the modulation is determined to be effective (Y), the location of the focused ultrasound treatment is retained for subsequent focused ultrasound treatments at 610 and the method terminates. If not (N), the selected location is determined to be ineffective, and the method returns to 602 to select a new location within the region of interest as a target for focused ultrasound treatment. The location of the focused ultrasound treatment target is personalized and can be modified live during treatment from acute feedback, or for subsequent treatments. The dosing of the focused ultrasound treatment can also be adjusted.

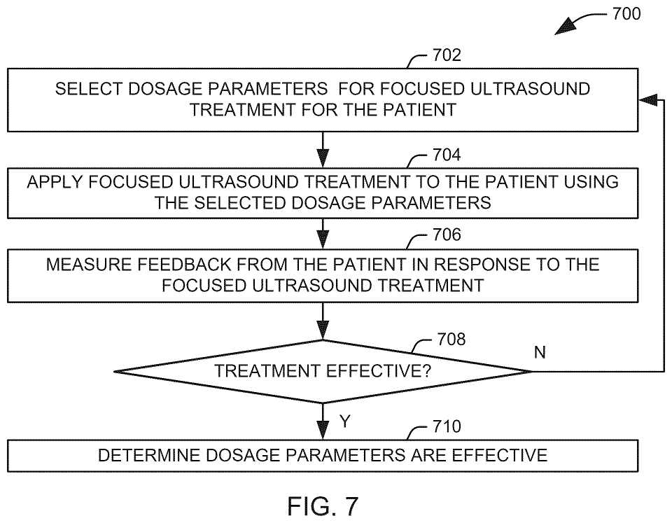

FIG. 7 illustrates a method 700 for selecting a dose for focused ultrasound treatment. It will be appreciated that the dose for focused ultrasound treatment is defined by parameters such as a frequency of application of the focused ultrasound treatment, an energy profile of the focused ultrasound treatment, a shape of the energy field, a direction of the energy field, a pulse rate of the focused ultrasound, a duration of discrete applications of energy, a number of discrete applications of energy, a total duration of the treatment, a type or dosage of a therapeutic provided in concert with the treatment, and an intensity, pressure, or power of the treatment. At 702, an initial dose for the modulation can be selected. It will be appreciated that the initial dose can be standard across patients, but in the illustrated implementation, the initial dose is patient-specific and can be determined according to factors such as a severity of the disorder, a location of the region of interest, and the type of tissue targeted.

At 704, focused ultrasound treatment is applied to the selected location and feedback from the patient in response to the applied focused ultrasound treatment is measured at 706. The feedback can include observations of a clinician on the appearance and behavior of the patient, self-reporting from the patient about symptoms of the disorder, measured electrical activity in the region of interest, and biometric parameters, such as those described in Tables II-VIII above. In particular, imaging may be used to determine the effectiveness and potential side effects of a given treatment. Acoustic feedback can also be used in focused ultrasound applications to evaluate both safety and effectiveness of the treatment.

At 708, an effectiveness of the focused ultrasound treatment is determined according to the measured feedback. This can be done, for example, either by a rule-based approach or by providing the measured feedback parameters to a predictive model. If the modulation is determined to be effective (Y), the selected dosage parameters for the focused ultrasound treatment are retained for subsequent application of energy at 710 and the method terminates. If not (N), the selected dosage parameters are determined to be ineffective, and the method returns to 702 to select new dosage parameters for treatment.

Once the treatment has been applied, feedback is collected from the patient to determine if the treatment has been effective. It will be appreciated that this can be done during or immediately after treatment (“acute feedback”), a short time (e.g., five hours to five days) after a treatment (“subacute feedback”) or a longer time (e.g., more than five days after a treatment (“chronic feedback”). The feedback can be acquired, for example, as any of the parameters listed in Tables II-VIII. To collect acute feedback, the tasks can be presented during or immediately after treatment to determine whether the patient's performance increases, decreases or otherwise changes in response to the treatment. The collected feedback can also include self-reporting from the patient, observations by clinicians, measured biometric parameters, such as heart rate variability and blood pressure, and other relevant parameters. In general, the parameters can be adapted based on review by a clinician, a rule-based system that generates suggested changes based on the initial parameters and the measured feedback, or via a predictive model trained on feedback data, treatment parameters, and clinical outcomes for previous patients.

Acute and subacute feedback from presentation application of a treatment can be obtained, for example, by measuring physiological parameters. In some examples, the physiological parameter can be a response of the patient's autonomic nervous system to focused ultrasound treatment and multiple physiological parameters can be measured during any given assessment session. The physiological parameters can be measured via a wearable device such as a ring, watch, or belt or via a smart phone or tablet, for example, in a naturalistic non-clinical setting such as when the patient is at home, work or other non-clinical setting. Exemplary physiological parameters include heart rate, heart rate variability, perspiration, salivation, blood pressure, pupil size, changes in pupil size, eye movements, brain activity, electrodermal activity, body temperature, and blood oxygen saturation level. Table III, above, provides non-limiting examples of physiological parameters that can be measured and exemplary tests to measure the physiological parameters. Sleep parameters can also be measured using wearable devices.

Particularly in treating neurocognitive disorders, cognitive parameters can be assessed by a battery of cognitive tests that measure, for example, executive function, decision making, working memory, attention, and fatigue. Table II, above, provides non-limiting examples of cognitive parameters that are gamified and that can be measured and exemplary methods and tests/tasks to measure such cognitive parameters. These cognitive tests can be administered in a clinical/laboratory setting or in a naturalistic, non-clinical setting such as when the user is at home, work, or other non-clinical setting. A smart device, such as a smartphone, tablet, or smart watch, can facilitate measuring these cognitive parameters in a naturalistic, non-clinical setting. For example, the Erikson Flanker, N-Back and Psychomotor Vigilance Tasks can be taken via an application on a smart phone, tablet, or smart watch.

Behavioral and psychosocial parameters, such as those described in Table VIII above, can measure the user's functionality, such as the user's movement via wearable devices as well as subjective/self-reporting questionnaires. The subjective/self-reporting questionnaires can be collected in a clinical/laboratory setting or in a naturalistic, in the wild, non-clinical setting such as when the user is at home, work, or other non-clinical setting. A smart device, such as a smartphone, tablet, or personal computer can be used to administer the subjective/self-reporting questionnaires. Using embedded accelerometers and cameras, these smart devices can also be used to capture the user's movements as well as facial expression analysis to analyze the user's facial expressions that could indicate mood, anxiety, depression, agitation, and fatigue. A wearable or portable device can also be used to measure parameters representing sleep length, sleep depth, a length of a sleep stage, and heart rate variability.

Treatment provided to the patient is adjusted based upon measured feedback. In general, the treatment can be adapted based on review of the feedback by a clinician, a rule-based system that generates suggested changes based on the initial parameters and the measured feedback, or via a predictive model trained on feedback data, treatment parameters, and clinical outcomes for previous patients. The feedback after focused ultrasound treatment can influence whether further focused ultrasound treatment is provided and how the provided focused ultrasound treatment should be adjusted. In terms of adjusting therapy in the context of focused ultrasound treatment, methods can involve adjusting the parameters or dosing of the focused ultrasound treatment such as, for example, the duration, frequency, or intensity of the focused ultrasound treatment. When the collected data indicates that the patient's condition has not improved, a method can involve adjusting the focused ultrasound treatment so that the focused ultrasound treatment is more effective. For example, if the patient was previously having focused ultrasound (FUS) delivered for five minutes during a therapy session, the patient can have the FUS subsequently delivered for twenty minutes during each session or if the patient was having FUS delivered every thirty days, the patient can have FUS subsequently delivered every two weeks. Conversely, if the parameter measurements indicate improvement, the focused ultrasound treatment parameters may not need adjustment and subsequent focused ultrasound treatment sessions can serve primarily as maintenance sessions or the intensity, frequency or duration of the focused ultrasound treatment can be decreased, for example. The above scenarios are only exemplary and are provided to illustrate that the presence and type of change of the patient's physiological parameter measurement values during and after therapy can influence whether the therapy should be adjusted or terminated.

Further, the degree of the patient's physiological, cognitive, psychosocial, or behavioral parameter measurement value during or after therapy can influence the parameters of subsequent focused ultrasound treatment. For example, if the specific patient seeking therapy has a physiological, cognitive, psychosocial, or behavioral parameter measurement value during or after treatment that is higher than the average parameter measurement value of the same patient population, the therapy can be more aggressive subsequently. Conversely, if the specific patient's parameter measurement value during or after treatment is lower than the average parameter measurement value of the same patient population, the therapy can be less aggressive subsequently. In other words, the severity or degree of the patient's physiological, cognitive, psychosocial, or behavioral parameter measurement value during or after focused ultrasound treatment (as well as baseline values and levels) can correlate to the degree or aggressiveness of future focused ultrasound treatment. The above scenarios are only exemplary and are provided to illustrate that the degree of change of the patient's physiological parameter measurement values during and after focused ultrasound treatment can influence the parameters of subsequent therapy.

In certain aspects, acute, subacute, and chronic feedback each determined from one or more combinations of a physiological, a cognitive, a psychosocial, and a behavioral parameter of the patient after a treatment. For example, obtaining a measurement of baseline values of one or more combinations of a physiological, a cognitive, a psychosocial, and a behavioral parameter of the patient can be obtained. The patient can then be exposed to an initial focused ultrasound signal to a neural target site of the patient. A subsequent measurement can be obtained of resultant values of the one or more combinations of the physiological, the cognitive, the psychosocial, and the behavioral parameter of the patient during or after application of the initial focused ultrasound signal. The resultant values can be compared to the baseline values to determine if the patient's cognitive and/or behavioral functions has improved. The focused ultrasound treatment can be adjusted upon a determination that the patient's cognitive and/or behavioral functions has not improved. For example, if it is determined that the focused ultrasound treatment was not successful, the focused ultrasound treatment can be provided to a different target location.

Form Factor of FUS Delivery and/or Sensors

An ultrasound device and/or sensor(s) can be incorporated into a wearable accessory item, such as a necklace or collar. For example, a necklace or collar can be configured to include at least one ultrasound transducer for delivering a focused ultrasound signal to a particular region of a subject's neck (e.g., an anterior or posterior region thereof) depending upon the desired neuromodulatory effect. The necklace or collar can additionally include an integral power source 34 (e.g., a rechargeable battery). The ultrasound transducer(s) can alternatively be powered by a wireless power source. The necklace or collar can be configured to obtain a pre-selected position about a subject's neck by, for example, using a positioning guide, or weighting the necklace or collar. Alternatively, the subject can manually adjust the necklace or collar as needed to optimize delivery of the ultrasound signal from the ultrasound transducer(s) to the ANS target site. In another example, an ultrasound device (and/or sensor) can be incorporated into a pillow. In some instances, the pillow can be configured as a collar for use in a reclined or upright position, such as on an airplane, in a car, on a couch, etc. The pillow can include at least one ultrasound transducer configured to deliver an ultrasound signal to an ANS target site (e.g., in a subject's head or neck). The pillow can also include a power source, which may be integrally connected with the pillow or located remotely (i.e., wirelessly) therefrom. Ultrasound devices (and sensors) can be incorporated into another wearable item, accessory, or article of clothing; or an object, device, or apparatus that a subject can use and, during use, comes into close or direct contact with a portion of the subject's body (e.g., the subject's neck). Examples of such transcutaneous ultrasound devices can include vests, sleeves, shirts, socks, shoes, underwear, belts, scarves, wrist bands, gloves, earpieces, band-aids, turtlenecks, pendants, rings, buttons, earrings, stickers, patches, bio-films, chairs, computers, beds, head rests (e.g., of a chair or car seat), cell phones, and the like.

As such, devices for transcutaneous FUS neuromodulation can have a variety of configurations. In some instances, a transcutaneous FUS delivery device may be configured as a belt or strap having at least one ultrasound transducer operably attached thereto. A FUS delivery device can be incorporated into clothing (such as, for example, a neck collar, brace, sweater, shirt, pants, socks, glove, stocking, skirt, shoes, underwear, vest, necklace, scarf, wrist band, waist band, ring, other jewelry, sportswear, earpieces, adhesive patches, or stickers. In addition, a FUS device can be embedded in a pillow, a bed, a head rest of a chair, a car seat, a car neck rest, a computer console, and other types of furniture. These devices can provide either ultrasound transducers for delivering a therapy signal, sensors, or a combination of both.

In certain instances, the ultrasound device can be incorporated into a diaper adapted for application on a patient's bladder. For example, the diaper can be configured for application to an the ventral part of an animal's (e.g. a dog or cat) bladder or a human's bladder for treatment of urinary incontinence for example. Alternatively, the ultrasound device could be incorporated into an ankle strap for treatment of urinary incontinence, for example.

### Chronic or Refractory Rhinitis

In certain aspects, a method and system for improving chronic or refractory rhinitis is provided. Target sites include nerve structures associated with the PPG such as, example, the SPG, the vidian nerve, or a sphenopalatine nerve. Feedback parameters include a sensed physiological parameter (e.g., a symptom) or a related physiological parameter indicative of the extent of the chronic or refractory rhinitis being treated. For example, changes in inflammatory cells (such as eosinophils, neutrophils, macrophages or lymphocytes), proteins, cytokines, chemokines, intraluminal gases, histamine, leukotrienes, nitric oxide (and its synthases and metabolites), or other markers/constituents of innate or adaptive mucosal or systemic immunity may be monitored/detected and the system/treatment may be adjusted to address these changes. Also, changes in temperature, pH, mucosal edema, changes related to mucosal remodeling (e.g., basement membrane thickness, epithelial damage), venous congestion, mucosal blood flow, mucosal hydration and rhinorrhea may be monitored or detected. In other instances, physiological parameters can include locoregional or systemic conditions or changes, such as other gross or molecular changes characteristic of chronic or refractory rhinitis. Example of such molecular changes can include up-regulation and/or down-regulation of various proteins (or their receptors). Such proteins may include, but are not limited to: interferon-alpha; interferon-gamma; interleukins (IL), such as IL-1-beta, IL-2, IL3-, IL-4, IL-5, IL-6, IL-7, IL-8, IL-10, IL-13, IL-15 and IL-17; growth-related oncogene-alpha; epithelial cell-derived neutrophil attractant-78; granulocyte chemotactic protein-2; eotaxin; released upon activation T-cell secreted (RANTES); thymus and activation-regulated chemokine (TARC), matrix metalloproteinases; vascular cell adhesion molecule-1; tumor necrosis factor-alpha; transforming growth factor-beta; chemokines (such as CCL13, CCL2, CCL8, CCL11, CCL18, CCL22, CXCL13); immunoglobulins; toll-like receptors; G-CSF; GM-CSF; MIP-1; VEGF; EGF; HGF; or other protein markers of inflammation. In addition, the inflammatory cell profile of the nasal mucosa may be monitored. For instance, relative or absolute eosinophil, neutrophil, macrophage, or lymphocyte (Th1, Th2, Th17) counts may be determined. Alterations in dendritic cells or associated proteins may also be used. Other markers may include nitric oxide and/or its synthases or metabolites, oxygen tension, and markers of ciliary dysfunction or defects in mucociliary flow. Microbes and/or their byproducts may also be used as markers. For example, bacteria such Staphylococcus aureus or Pseudomonas aeruginosa, fungi, or viruses as well as by-products of these or other organisms may be used. Additionally, gene transcripts, protein markers, or markers of microbial biofilms may be used.

### Chronic Rhinosinusitis

Chronic rhinosinusitis or “CRS” can refer to the disease entity characterized by inflammation of the nasal cavity and/or paranasal sinuses lasting greater than about twelve weeks duration. Symptoms may include, but are not limited to: facial pain or pressure; nasal congestion or fullness; nasal obstruction or blockage; nasal discharge (rhinorrhea or post-nasal drip); hyposmia/anosmia; and purulence in the nasal cavity. Other potential symptoms include: headache; fever; halitosis; fatigue; dental pain; cough; and ear pain/pressure/fullness. In one example, the presence of severe inflammation and irritation with thickened discolored or purulent discharge can be indicative of CRS, whereas pale mucosa with clear discharge can be suggestive of allergic rhinitis. In another example, CRS can refer to recalcitrant forms of the disease in which symptoms persist despite medical or surgical treatment, as well as instances where patients cannot receive standard medical or surgical care due to contraindications for such care.

Target sites include target location is a nerve structure associated with the pterygopalatine fossa (PPF), such as, for example, a sphenopalatine ganglion (SPG), a vidian nerve, or a sphenopalatine nerve.

Feedback parameters include physiological parameters such as a characteristic, symptom, molecule, or function of the body that is associated with CRS. Examples of such physiological parameters can include, but are not limited to, mucosal blood flow, sinusoidal filling, mucosal thickness, mucosal secretion, protein or chemical concentrations (e.g., cytokines, histamines), pressure (e.g., sinonasal intraluminal pressure), temperature, pH, mucosal thickness, changes related to mucosal remodeling (e.g., basement membrane thickness, epithelial damage), electrochemical gradients, microbial products and byproducts, gases (e.g., nitric oxide), as well as other locoregional or systemic conditions or changes, such as other gross or molecular changes characteristic of CRS. Examples of such molecular changes can include up-regulation and/or down-regulation of various proteins (or their receptors). Such proteins may include, but are not limited to: interferon-alpha; interferon-gamma; interleukins (IL), such as IL-1-beta, IL-2, IL3-, IL-4, IL-5, IL-6, IL-7, IL-8, IL-10, IL-13, IL-15 and IL-17; growth-related oncogene-alpha; epithelial cell-derived neutrophil attractant-78; granulocyte chemotactic protein-2; eotaxin; released upon activation T-cell secreted (RANTES); thymus and activation-regulated chemokine (TARC), matrix metalloproteinases; vascular cell adhesion molecule-1; tumor necrosis factor-alpha; transforming growth factor-beta; chemokines (such as CCL13, CCL2, CCL8, CCL11, CCL18, CCL22, CXCL13); immunoglobulins; toll-like receptors; G-CSF; GM-CSF; MIP-1; VEGF; EGF; HGF; or other protein markers of inflammation.

In addition, the inflammatory cell profile of the sinonasal mucosa can be monitored as part of feedback system or method. For instance, relative or absolute eosinophil, neutrophil, macrophage, or lymphocyte (Th1, Th2, Th17) counts may be determined. Alterations in dendritic cells or associated proteins may also be used. Other markers may include nitric oxide and/or its synthases or metabolites, oxygen tension, and markers of ciliary dysfunction or defects in mucociliary flow. Microbes and/or their byproducts may also be used as markers. For example, bacteria such Staphylococcus aureus or Pseudomonas aeruginosa, fungi, or viruses as well as by-products of these or other organisms may be used. Additionally, gene transcripts, protein markers, or markers of microbial biofilms may be used.

### Autonomic Instability/Autonomic Dysfunction

Autonomic instability or autonomic dysfunction can refer to an abnormal fluctuation in sympathetic and/or parasympathetic tone above or below a normal or baseline (e.g., healthy) level. Such abnormal fluctuation can be chronic (e.g., months, years, etc.) or acute (e.g., hours, days, etc.). A medical condition associated with autonomic instability or a medical condition associated with autonomic dysfunction can refer to a disease, disorder, sign, or symptom that is associated with, or results at least in part from, autonomic instability or dysfunction. In some instances, a medical condition associated with autonomic instability or dysfunction can include neurodegenerative disease, such as Parkinson's disease, multiple systems atrophy, other atypical parkinsonian syndromes, neuropathy, inflammatory/autoimmune disease, demyelinating disorders, such as Guillian Barre Syndrome, parancoplastic syndromes, infectious ctiologies (e.g., botulism), Chagas Disease, addictions (e.g., alcoholism), mitochondrial disorders, spinal cord injury, brain injury, concussions, other trauma, complex regional pain syndrome, heavy metal toxicity, other toxicities, amyloidosis, porphyria, and also inherited disorders such as Fabry's disease and Hereditary Sensory Autonomic Neuropathy syndromes. In other instances, a medical condition associated with autonomic instability or dysfunction can be characterized by one or more signs or symptoms, such as problems with blood pressure regulation (e.g., orthostatic hypotension and hypertension), cardiac arrhythmia (e.g., tachycardia/bradycardia), dysfunctional gastrointestinal motility (e.g., reflux, gastroparesis), genitourinary trouble (e.g., urinary incontinence, overactive bladder, erectile dysfunction), impaired sweat response (e.g., hyperhidrosis or anhidrosis), dysfunctional temperature regulation, and also changes in salivary production (e.g., sialorrhea), trouble with ambulation due to lightheadedness/near syncope, recurrent syncope, severe reflux, trouble with swallowing, difficulty clearing oral secretion, reoccurring aspiration, poor gastrointestinal absorption, reoccurring bowel obstructions, unexplained fevers, constant sweating, hot/cold intolerance, frequent urination, urinary incontinence, and recurrent urinary tract infections.

Target sites to improve autonomic instability/autonomic dysfunction include a sympathetic chain ganglion, an efferent of a sympathetic chain ganglion, or an afferent of a sympathetic chain ganglion. A sympathetic chain ganglion can be a cervical sympathetic ganglion, a thoracic sympathetic ganglion, or a stellate ganglion. Examples of cervical sympathetic ganglia can include an upper cervical sympathetic ganglion, a middle cervical sympathetic ganglion, or a lower cervical sympathetic ganglion. Examples of thoracic sympathetic ganglia can include a T1 sympathetic ganglia, a T2 sympathetic ganglia, a T3 sympathetic ganglia, a T4 sympathetic ganglia, a T6 sympathetic ganglia, or a T7 sympathetic ganglia.

Feedback parameters include a physiological characteristic, sign, symptom, or function associated with autonomic instability or a medical condition associated therewith, such as a chemical moiety or nerve activity (e.g., electrical activity). Examples of such chemical moieties and nerve activities can include the activity of autonomic ganglia (or an autonomic ganglion), protein concentrations, electrochemical gradients, hormones (e.g., cortisol), neuroendocrine markers, such as corticosterone, norepinephrine and melatonin, electrolytes, laboratory values, vital signs (e.g., blood pressure), markers of locomotor activity, cardiac markers (e.g., EKG RR intervals), or other signs and biomarkers associated with autonomic instability or a medical condition associated therewith.

### Functional Gastrointestinal Disorders

A functional gastrointestinal disorder can refer to a disease or condition having one or more gastrointestinal (GI) symptoms or combinations of GI symptoms of a chronic or recurrent nature that do not have an identified underlying pathophysiology (e.g., are not attributable to anatomic or biochemical defects). In the absence of an objective marker(s), the identification and classification of functional GI disorders can be based on symptoms. Examples of such symptoms can include abdominal pain, early satiety, nausea, bloating, distention, and various symptoms of disordered defecation. In some instances, such classification can be based on the Rome diagnostic criteria. Non-limiting examples of functional GI disorders can include visceral pain, irritable bowel syndrome (IBS), functional dyspepsia, functional constipation, functional diarrhea, gastroesophageal reflux disease (GERD), and functional abdominal bloating. Functional GI disorders (FGIDs) represent a highly prevalent group of heterogeneous disorders, and their diagnosis is based on symptoms in the absence of a reliable structural or biochemical abnormality as noted previously. IBS, for example, is a disorder that leads to debilitating symptoms that include abdominal pain, cramping, discomfort, bloating and changes in bowel movements (diarrhea, constipation or alternating diarrhea/constipation). In patients with IBS, heightened pain sensitivity is observed in response to experimental visceral stimulation, and such patients are said to have visceral pain hypersensitivity.

Examples of FGIDs are listed above and can also include: functional esophageal disorders (e.g., functional heartburn, functional chest pain of presumed esophageal origin, functional dysphagia and globus); functional gastroduodenal disorders, such as functional dyspepsia (e.g., postprandial distress syndrome and epigastric pain syndrome), belching disorders (e.g., aerophagia and unspecified excessive belching), nausea and vomiting disorders (e.g., chronic idiopathic vomiting, functional vomiting, and cyclic vomiting syndrome), and rumination syndrome; functional bowel disorders, such as unspecified functional bowel disorder; functional abdominal pain syndrome; functional gallbladder and Sphincter of Oddi (SO) disorders (e.g., functional gallbladder disorder, functional biliary SO disorder, and functional pancreatic SO disorder); functional anorectal disorders, such as functional fecal incontinence, functional anorectal pain (e.g., chronic proctalgia and proctalgia fugax), and functional defecation disorders (e.g., dyssynergic defecation and inadequate defecatory propulsion); childhood functional GI disorders in infants/toddlers, such as infant regurgitation, infant rumination syndrome, cyclic vomiting syndrome, infant colic, functional diarrhea, infant dyschezia and functional constipation; and childhood functional GI disorders in children/adolescents, such as vomiting and aerophagia (e.g., adolescent rumination syndrome, cyclic vomiting syndrome, and aerophagia), abdominal pain-related functional GI disorders (e.g., functional dyspepsia, IBS, abdominal migraine, and childhood functional abdominal pain syndrome), and constipation and incontinence (e.g., functional constipation and non-retentive fecal incontinence). Subjects treatable by the present disclosure can, in some instances, be diagnosed with (or suspected of having) a functional GI disorder as well as one or more related or unrelated medical conditions. Table IX provides examples of functional GI disorders

TABLE IX
GI Disorder associated symptom

Irritable Bowel Syndrome (IBS) [symbol]
pain associated with IBS
GI discomfort associated with IBS
abnormal bowel habits associated with IBS [symbol]
IBS
constipation predominant IBS (C-IBS)
diarrhea predominant IBS (D-IBS) [symbol]
constipation/diarrhea [symbol] (C/D-IBS) [symbol]
IBS
Functional [symbol] (FD)
gastric distress associated with FD
abdominal pain associated with FD
bloating associated with FD
GI discomfort associated with FD [symbol]
associated with FD
Functional Abdominal Pain Syndrome [symbol]
Disorder ( [symbol] )

[symbol]

[symbol]

syndrome [symbol]
pain syndrome
Functional [symbol]
non-cardiac chest pain (e.g., abnormal [symbol] of [symbol] or [symbol] )
functional heart burn
functional [symbol] and [symbol]
IBS Secondary to Chron's Disease
CD-IBS, pain, and [symbol] occurs during [symbol] [symbol]
Syndrome
Nausea and Vomiting Disorders
chronic [symbol] vomiting syndrome
functional vomiting syndrome
cyclic vomiting syndrome
Functional Gallbladder and [symbol] ( [symbol] ) Disorders
functional gallbladder disorder
functional [symbol] disorder
Functional [symbol] Disorders
functional fecal incontinence
Functional [symbol] Pain

[symbol]

[symbol]

Childhood Functional GI Disorders in Infants/Toddlers
infant [symbol]
infant [symbol]
cyclic vomiting syndrome [symbol]
indicates data missing or illegible when filed

Targets sites include a ganglion of the SNS including a sympathetic chain ganglion, an efferent of a sympathetic chain ganglion, or an afferent of a sympathetic chain ganglion. The sympathetic chain ganglion can be a cervical sympathetic ganglion, a thoracic sympathetic ganglion, or a stellate ganglion. Examples of cervical sympathetic ganglia can include an upper cervical sympathetic ganglion, a middle cervical sympathetic ganglion, or a lower cervical sympathetic ganglion. Examples of thoracic sympathetic ganglia can include a T1 sympathetic ganglia, a T2 sympathetic ganglia, a T3 sympathetic ganglia, a T4 sympathetic ganglia, a T6 sympathetic ganglia, or a T7 sympathetic ganglia. Other examples of ANS nerve targets can include a mesenteric plexus or a gastric plexus.

Feedback parameters include a physiological characteristic, sign, symptom, or function associated with the functional GI disorder, such as a chemical moiety or nerve activity (e.g., electrical activity). Examples of such chemical moieties and nerve activities can include the activity of autonomic ganglia (or an autonomic ganglion), the activity of a spinal cord segment or spinal nervous tissue associated therewith, protein concentrations (e.g., BDNF, IL-1B, KC/GRO, NGAL, TIMP-1, TWEAK, etc.), electrochemical gradients, hormones, neuroendocrine markers (e.g., corticosterone and norepinephrine), electrolytes, laboratory values, vital signs (e.g., blood pressure), markers of locomotor activity, inflammatory markers, or other signs and biomarkers associated with functional GI disorders.

### Inflammatory Disorders

An inflammatory disorder is a disorder in which a break in homeostasis or a failure to restore homeostasis causes excessive recruitment and activation of innate (and occasionally adaptive) immune cells often without physiological resolution (e.g. chronic inflammation). Symptoms of an inflammatory disorder include, for example, chronic pain, redness, swelling, stiffness, or damage to normal cells. Inflammatory disorders include disorders caused by a defective, excessive or non-resolving acute inflammatory reaction such as, for example, serous inflammation, fibrinous inflammation, suppurative or purulent inflammation, or leukocyte-induced injury. Inflammatory disorders also include disorders caused by defective or excessive chronic inflammation such as, for example, ulcers, granulomatous inflammation or leukocyte-induced injury. Non-limiting examples of inflammatory disorders are atherosclerosis; inflammatory bowel diseases, such as Crohn's disease and ulcerative colitis; non-allergic asthma; acute respiratory distress syndrome (ARDS); acute lung injury (ALI); chronic respiratory distress syndrome (CRDS); dermatitis; gout; eczema, gastritis; hepatitis; prostatitis; sinusitis; splenitis; nephritis; inflammation of body organs including joint inflammation including joints in the knees, limbs or hands; and neurogenic lung edema.

In certain embodiments, a method of improving an inflammatory disorder in a subject involves positioning an ultrasound device on a neural target site of a maladaptive sympathetic reflex or disturbance of the subject. A maladaptive sympathetic reflex or disturbance is imbalanced sympathetic nerve activity that enters the lymphatic organs (e.g., spleen, bone marrow); endocrine organs such as the liver or adrenal gland; or immune system organ or immune systems cells. A maladaptive sympathetic reflex or disturbance causes abnormal immune activity such as decreased, increased, imbalanced, or impaired immune activity as compared to the immune activity of a healthy subject. A healthy subject is a subject who has not previously been diagnosed as having any signs or symptoms indicating the presence of immune system dysfunction, a history of immune system dysfunction, or evidence of immune system dysfunction. A healthy subject is also a subject who, if examined by a medical professional, would be characterized as free of immune system dysfunction.

A maladaptive sympathetic reflex or disturbance can be disinhibited sympathetic nerve activity that enters the lymphatic organs or immune system organs. A maladaptive reflex or disturbance includes a sympathetic anti-inflammatory reflex, Sympathico-Babinski, disinhibited spinally generated sympathetic activity, and paroxysmal sympathetic activity. These conditions generally act on the ANS causing a systemic immune-suppression or immune-activation. A maladaptive sympathetic reflex or disturbance can be present in a subject with a hyperactive or hypoactive immune system. Further, a maladaptive sympathetic reflex or disturbance can be present after CNS injury including injury to the brain, such as stroke, TBI, and subarachnoid heamorrhage (SAH); SCI; and other CNS or spinal cord or nervous system damage/injury, such as damage to nerves. Injury can be due to acute or chronic degenerative conditions. Chronic degeneration conditions include injury to the brain or spinal cord by compressive tumor growth but also degenerative myelopathy and neurodegenerative disease.

Under physiological conditions, excitatory sympathetic signals are controlled by supraspinal inhibition. After SCI, loss of supraspinal control leads to spinally generated SNA. SNA originates below SCI injury and leads to maladaptive efferent sympathetic activity signaling to the spleen, via the splenic or splanchnic nerve and directly to the adrenal gland. Such activity can culminate in large excitatory spinal sympathetic reflexes. Spinal sympathetic reflexes occur in analogy to pathological Babinski motor reflexes, which are caused by a loss of supraspinal (bulbospinal) tonic inhibition (“Sympathetic-Babinski”). Again, without wishing to be bound by theory, disinhibited spinal generated SNA after high thoracic (Th3) SCI can lead to ad hoc induction of excitatory SNA burst entering the splenic nerve and adrenal gland associated with increased levels of norepinephrine (NE) in the circulation and the spleen, which in turn causes apoptosis of immune cells in the spleen and other lymphoid organs. This results in a decrease of spleen size, partial loss of immune cells and an elevated susceptibility to infection. Blocking SNA signaling (“shielding”) by preceding peripheral denervation of the splenic nerve ameliorates functional SCI-IDS and bacterial load after Th3-SCI. Moreover, the peripheral splenic nerve can be a target for immunomodulation after SCI in order to restore impaired host defense after SCI (SCI-IDS) by blocking SNA to the spleen. As such, spleen and adrenal gland shielding can be an interventional strategy to ameliorate functional SCI-IDS to prevent infections in patients at risk.

Again, without wishing to be bound by theory, it is believed that enhancing the maladaptive sympathetic reflex or disturbance may also artificially immune-suppress a patient and may attenuate immune diseases, including inflammatory disorders affecting all systems of the body. Those systems include, but are not limited to, the nervous system (peripheral and central), bone, cartilage, bronchial system/lung, pancreas, liver, and hematological systems. Blocking sympathetic activation reactive to stress responses may harness a patient's immune defense system against cancer. Other conditions causing immune suppression and elevated risk for infections are listed in Table II below.

[symbol]

Term

[symbol]

progenic
tonic [symbol] seizures
autonomic seizures with [symbol] [symbol] eizures [symbol]
(autonomic) seizures/epilepsy/fits
autonomic sympathetic seizures
Structural
brain stem attack
hypothalamic-midbrain dysregulation syndrome
acute midbrain syndrome
acute hypothalamic instability
hypothalamic storm
Clinically Descriptive [symbol]
hypertension associated with [symbol]
(central) autonomic dysfunction/syndrome [symbol]
syndrome
neuroleptic malignant-like syndrome
autonomic storms/storming
autonomic hyperactivity

[symbol]

(central) autonomic instability [symbol]
with sustained muscle contraction
autonomic dysregulation [symbol]
sympathetic storms
sympathetic storms/storming
autonomic disorder (and spasticity)
neuro storm [symbol]
autonomic instability with dystonia [symbol]
state [symbol]
autonomic instability [symbol]
crises
arc de [symbol] and [symbol]
storming: transient autonomic dysfunction [symbol]
sympathetic hyperactivity
hypermetabolic [symbol] [symbol]
indicates data missing or illegible when filed

In certain embodiments, the ultrasound signal blocks neural conduction in the neural target site. Such blocking of neural conduction can reduce or balance the patient's sympathetic tone to improve the patient's immune response. In certain embodiments, the patient is suffering from sepsis, a central nervous system injury, antibiotic resistance, compensatory anti-inflammatory response syndrome (CARS), chronic inflammatory response syndrome (CIRS), CNS injury-induced immune deficiency syndrome (CUDS), including stroke-induced immune deficiency syndrome (SIDS), SCI-IDS, traumatic brain injury-induced immune depression syndrome (TBI-IDS), or other immune deficiencies.

In certain embodiments, the ultrasound signal stimulates neural conduction in the neural target site. Such stimulation can enhance the patient's sympathetic tone to immune-suppress the patient, suppress the patient's baseline sympathetic tone to suppress disease-associated increases in immune function, or increase activity of the patient's intact sympathetic circuitry to suppress the patient's immune function. Such stimulation can be used in circumstances where the patient has undergone organ transplantation (including bone marrow transplantation); suffers from an autoimmune disorder, including multiple sclerosis, rheumatoid arthritis, myasthenia gravis or myositis; suffers from corticosteroid side effects including osteoporosis or cortisone-induced psychosis; or suffers from other adverse effects from immune suppressive therapies. Other conditions include hyper-immune disorders including multiple sclerosis, rheumatoid arthritis, and allergic conditions. Conditions also include hypo-active immune system disorders such as cancers or infections.

In certain embodiments, a method of improving an inflammatory disorder includes improving tolerability of long-lasting immune modulatory therapy. Such a method can be used when orthodox immune modulatory treatment (e.g. steroids) cannot be provided because of limiting side effects of such treatment. Such side effects include, for example, osteoporosis and psychosis. As such, an exemplary method involves a corticosteroid sparing approach, which allows for better treatment tolerability. This can be advantageous in chronically ill patients when long lasting therapy regimens are required. In such embodiments, the patient population is patients that cannot tolerate corticosteroids or other pharmaceutical (e.g. drug) or biological agents to improve their inflammatory disorder. To not “tolerate” such agents, is meant that the side effects associated with the administration of such agents is greater than the therapeutic benefit gained by such agents. In certain embodiments, a method of improving an inflammatory disorder comprises positioning a FUA device in communication with a neural target site that contributes to immune activity of a subject. The method further includes activating the FUS device to deliver a therapy signal to the neural target site and improving the subject's inflammatory disorder. The subject's inflammatory disorder is improved without the administration of corticosteroids or other pharmaceutical agents that are indicated for treating or otherwise improving inflammatory disorders.

Target sites include a PNS structure anatomically relevant to maladaptive sympathetic reflexes or disturbances. Preferably, the neural target site innervates an endocrinological or lymphatic tissue or organ involved in the immune response and inflammatory response and state of the patient. In certain embodiments, the neural target site is a neural target site of the ANS., which includes the sympathetic nervous system (SNS) and the parasympathetic nervous system (PSNS). Such sites include autonomic nerves (including pre- and post-ganglionic fibers of the ANS), autonomic ganglia, and autonomic plexus. Preferably, the neural target site is a part of the SNS, such as a sympathetic ganglion, a sympathetic nerve or a sympathetic plexus. Regarding sympathetic ganglia, the neural target site can be a prevertebral ganglion or a paravertebral ganglion (sympathetic nerve chain ganglion) or the sympathetic trunk. Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus.

Target feedback parameters include physiological parameters associated with a deficient or abnormal immune system such as a hypoactive or hyperactive immune system. In some instances, such physiological parameters can include any characteristic or function associated with immune function, such skin temperature, protein concentrations, heart rate, blood pressure, biomarkers of the immune system or autonomic nervous system, electrochemical gradients, electrolytes, laboratory values, body temperature, and vital signs.

Sensors to measure physiological parameters can be external of the patient's body, or on the patient's body. The parameters can be measured, for example, from blood, saliva, or sweat biomarkers. Non-limiting examples of physiological parameters include vital signs; EEG, EMG; FNIRS; heart rate variability; immune and hormonal markers, IL-6, TNF, other inflammatory mediators; sedimentation rate such as ESR for example, CRP, CBC and differential ratios of cell; circadian rhythms; and other means of feedback linked to stimulation modulation. Physiological parameters that are biomarkers include but are not limited to BDNF, Neuropeptide Y, cortisol, orexin, oxytocin, epinephrine, melatonin norepinephrine, DHEA, IL-6, IL-1, IL-4, TNF. Other physiological parameters include eye tracking, pupil tracking, facial expression, thermography, and cognitive and behavioral measures such as stress, anxiety or hyperactivity.

With respect to determining a level of a physiological parameter indicative of immune activity, non-limiting physiological parameters include: leukocyte subset ratios, such as the CD4/CD8 ratio; immunoglobulin (antibody) levels or antigen-specific antibody levels (e.g., antibodies directed against specific proteins in the body); NK lysis; granulocyte, neutrophil or monocytic activation; levels of biomarkers of inflammation such as, for example, C-reactive protein; levels of biomarkers of infection such as, for example, procalcitonin; or levels of lymphocytes or leukocytes. The above-described physiological parameters are exemplary. Other physiological parameters can be detected depending on various factors. Such factors include, for example, the specific inflammatory disorder being treated, the subject's age, the body organs affected, the duration or phase of the disorder, and other factors.

Various assays, tests and procedures can be employed to determine the level of a physiological parameter indicative of immune activity. For example, flow cytometry analysis can be used to determine the ratio of leukocyte subsets, such as the CD4/CD8 ratio; blood analysis can be used to determine antibody levels or antigen-specific antibody levels; immune cell function assays (e.g., mitogen stimulation of lymphocytes with cytokine readouts or proliferation; NK cell lysis assays; or radiological testing, such as T1/T2-weighted imaging of the brain or spinal cord with gadolinium chloride enhancement to identify regions of vascular compromise which are either caused by aberrant inflammation or cause it to develop. The above-described assays, tests and procedures are exemplary. Other techniques can be used to determine the level of a physiological parameter indicative of immune activity depending on various factors. Such factors include, for example, the specific inflammatory disorder being treated, the subject's age, the body organs affected, the duration or phase of the disorder, and other factors.

The present disclosure provides another method for improving an inflammatory disorder in a subject. Such a method comprises determining the level of a physiological parameter that is indicative of inflammation of the subject and comparing the determined level of the physiological parameter with a control value. The method further includes placing an ultrasound delivery device into communication with a neural target site that contributes to the inflammatory disorder. The method further includes activating the ultrasound delivery device to deliver an ultrasound signal to the neural target site to improve the subject's inflammatory disorder.

In particular, after determining the level of the physiological parameter, the detected level can be compared to a control value to determine if the detected level is abnormal, thereby indicating that the subject suffers from an immune system dysfunction. Control values can be based upon the level of a corresponding physiological parameter obtained from a control population. The control population can be the general population or a select population. The select population can be a group of healthy subjects, a group of subjects that are at risk of an inflammatory disorder, or a group of subjects that suffer from an inflammatory disorder. For example, if the level of the physiological parameter is the units of a particular biomarker per ml of blood, the control value can be based upon the units of the particular biomarker per ml of blood in subjects of the general or select population.

As stated above, after comparing the detected level of the physiological parameter with the control value, a determination can be made as to whether the subject has an inflammatory disorder. In some instances, an increased level of the detected physiological parameter (as compared to the control value) can be indicated of an inflammatory disorder. In other instances, a decreased level of the detected physiological parameter (as compared to the control value) can be indicative of an inflammatory disorder. For example, CD4/CD8 ratios are often used as indicators of “adaptive immune health” with a normal ratio of greater than 2.0. Values lower than 2.0 are often used clinically as a diagnostic criteria for various suspected immune diseases including HIV, AIDS, anemia, multiple sclerosis, and chronic infections. A ratio higher 2.0 could indicate the presence of an infection. Furthermore, prolonged elevation of inflammatory markers, such as a C-reactive protein level greater than 5 mg/l, is often associated with exacerbated inflammatory disease; prolonged elevation of markers of infection, such as a procalcitonin level greater than 0.5 ng/ml or a leukocyte level greater than 9 nl, is often associated with exacerbated infectious disease; prolonged suppression of lymphocytes, such as a lymphocyte level less than 1.5/nl or a leukocyte level of less than 4/nl, is often associated with exacerbated immune-suppression or impaired host defense. Other indicators of an inflammatory disorder include the presence of pathogenic antibodies; excessive NK lysis; the presence of innate cell respiratory burst function such as granulocyte, neutrophil and monocytic activation; enhanced lesions indicating a break-down of the blood-brain barrier; or the presence of sustained leucopenia, leukocytosis, lymphopenia, lymphocytosis, monocytosis, monopenia, neutrophilia, or neutropenia. The above-described factors and values are exemplary and other factors and values can indicate whether a subject has an inflammatory disorder.

### Immune Disorders

Immune disorders include autoimmune disorders, hypersensitivity syndromes, immune deficiency disorders, and combinations thereof. Such immune disorders can be caused by cell-mediated immunity (T lymphocytes), humoral immunity (B lymphocytes) and immune tolerance. Immune disorders may result in destruction of body tissue, abnormal growth of an organ, and/or changes in organ function. An immune disorder may affect one or more organ or tissue types.

An autoimmune disorder is a type of immune disorder resulting from an abnormal or exaggerated adaptive immune response that targets healthy cells or tissues that should not normally cause an immune reaction in the body. Autoimmune disorders include disorders in line with Witebsky's Postulates. Areas often affected by autoimmune disorders include, for example, the blood and blood vessels; connective tissue; endocrine glands or hormone producing organs such as the thyroid, pancreas, or adrenal glands; joints; muscles such as the heart; red blood cells; eyes; the skin; the gastrointestinal (GI) or digestive system; the brain, spinal cord, central nerves and peripheral nerves; bone; reproductive tissues such as the ovaries and testes; and breast tissue.

In certain embodiments, the autoimmune disorder is multiple sclerosis, ankylosing spondylitis, rheumatoid arthritis, celiac disease, myositis, myasthenia gravis, Addison's disease, lupus, hemolytic anemia, vitiligo, scleroderma, psoriasis, Hashimoto's disease, Addison's disease, Grave's disease, reactive arthritis, Sjogren's syndrome, nephritis, chronic Lyme disease, vasculitis, endocarditis, alopecia areata, urticaria, vasculitis, uveitis, pemphigus, Fibromyalgia, thrombophelebitis, erythema nodusum, dermatitis, eczema, Type 1 Diabetes, temporal arteritis, Crohn's Disease, Behcet's disease, or psoriatic arthritis.

In further embodiments, the autoimmune disorder is multiple sclerosis, rheumatoid arthritis, lupus, celiac disease, Sjogren's syndrome, ankylosing spondylitis, Type 1 Diabetes, myositis, myasthenia gravis, alopecia areata, vasculitis, temporal arteritis, or eczema.

Hypersensitivity syndromes include immediate (Type I) hypersensitivity, antibody-mediated (Type II) hypersensitivity, immune complex-medicated (Type III) hypersensitivity, and cell-mediated (Type IV) hypersensitivity. In Type I hypersensitivity, the immune response releases vasoactive and spasmogenic substances that act on vessels and smooth muscle and releases pro-inflammatory cytokines that recruit inflammatory cells. In Type II hypersensitivity, secreted antibodies participate directly in injury to tissues by inducing inflammation. Antibodies may also interfere with cellular functions and cause disease without tissue injury. In Type III hypersensitivity, antibodies bind antigens and then induce inflammation directly or by activating complement. The leukocytes that are recruited (neutrophils and monocytes) produce tissue damage by the release of lysosomal enzymes and the generation of toxic free radicals. In Type IV hypersensitivity, sensitized T lymphocytes are the cause of the cellular and tissue damage.

Non-limiting examples of Type I hypersensitivity disorders are chronic or acute allergies, atopic forms of bronchial asthma, and anaphylaxis. Non-limiting examples of Type II hypersensitivity syndromes are autoimmune hemolytic anemia, autoimmune thrombocytopenic purpura, pemphigus vulgaris, vasculitis caused by antineutrophil cytoplasmic antibodies, Goodpasture syndrome, acute rheumatic fever, myasthenia gravis, Graves disease, insulin-resistant diabetes, and pernicious anemia. Type II hypersensitivity syndromes may be caused by the production of antibodies that bind to non-self antibodies, such as after an allogenic transplantation resulting in organ rejection; blood-group incomparability resulting in hemolysis; antibodies that bind to tumor-associated antigens resulting in paraneoplastic syndromes, neuropathies, and channelopathies, for example. Type II hypersensitivity may also be caused by antibodies directed against cell-membrane bound medications resulting in medication-induced cell death, such as heparin-induced thrombocytopenia, for example. Non-limiting examples of Type III hypersensitivity disorders are systemic lupus erythematosus, poststreptococcal glomerulonephritis, acute glomerulonephritis, serum sickness, Arthus reaction, reactive arthritis, and polyarteritis nodosa. Non-limiting examples of Type IV hypersensitivity syndromes are contact dermatitis, multiple sclerosis, type 1 diabetes, transplant rejection, rheumatoid arthritis, tuberculosis, and peripheral neuropathy.

Immune deficiency disorders include primary immunodeficiency disorders and secondary immunodeficiency disorders. Most primary immunodeficiency disorders are genetically determined and affect specific immunity (i.e. the humoral and cellar arms of adaptive immunity) or non-specific host defense mechanisms mediated by complement proteins and cells such as phagocytes or NK cells (innate immunity). Primary immunodeficiency disorders can be mediated by T-cell defects, B-cell defects or both T-cell and B-cell defects. Non-limiting examples of primary immunodeficiency disorders are X-linked agammaglobulinemia, common variable immunodeficiency, isolated IgA deficiency, hyper-IgM syndrome, DiGeorge syndrome, severe combined immunodeficiency disease (SCID), Wiskott-Aldrich syndrome, and genetic deficiencies of the complement system. Secondary immunodeficiency disorders are acquired and may arise as complications of infections; malnutrition; aging; or side effects of immunosuppression, irradiation, or chemotherapy for cancer and other autoimmune disorders.

Non-limiting examples of secondary immunodeficiency disorders are Acquired Immunodeficiency Syndrome (AIDS), human immunodeficiency virus (HIV) infection, combined immune deficiency syndrome (CIDS), and a spinal cord injury-induced immune depression syndrome (SCI-IDS).

In certain embodiments, a method of improving an immune disorder such as an autoimmune disorder, a hypersensitivity syndrome or an immune deficiency disorder, includes improving tolerability of long-lasting immune modulatory therapy. Such a method can be used when orthodox immune modulatory treatment (e.g. steroids) cannot be provided because of limiting side effects of such treatment. Such side effects include, for example, osteoporosis and psychosis. As such, an exemplary method involves a corticosteroid sparing approach, which allows for better treatment tolerability. This can be advantageous in chronically ill patients when long lasting therapy regimens are required. In such embodiments, the patient population is patients that cannot tolerate corticosteroids or other pharmaceutical (e.g. drug) or biological agents to improve their immune disorder. To not “tolerate” such agents, is meant that the side effects associated with the administration of such agents is greater than the therapeutic benefit gained by such agents. In certain embodiments, a method of improving an immune disorder comprises positioning an ultrasound delivery device in communication with a neural target site that contributes to immune activity of a subject. The method further includes activating the ultrasound delivery device to deliver an ultrasound signal to the neural target site and improving the subject's immune disorder. The subject's immune disorder is improved without the administration of corticosteroids or other pharmaceutical agents that are indicated for treating or otherwise improving immune disorders

In certain embodiments, the neural target site is a neural target site of the ANS, which includes the sympathetic nervous system (SNS) and the parasympathetic nervous system (PSNS). Such sites include autonomic nerves (including pre- and post-ganglionic fibers of the ANS), autonomic ganglia, and autonomic plexus. Preferably, the neural target site is a part of the SNS, such as a sympathetic ganglion, a sympathetic nerve or a sympathetic plexus. Regarding sympathetic ganglia, the neural target site can be a prevertebral ganglion or a paravertebral ganglion (sympathetic nerve chain ganglion). Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus.

Target feedback parameters include blood, saliva, or sweat biomarkers. Non-limiting examples of physiological parameters include vital signs; EEG, EMG; FNIRS; heart rate variability; immune and hormonal markers, IL-6, TNF, other inflammatory mediators; sedimentation rate such as ESR for example, CRP, CBC and differential ratios of cell; circadian rhythms; and other means of feedback linked to stimulation modulation. Physiological parameters that are biomarkers include but are not limited to BDNF, Neuropeptide Y, cortisol, orexin, oxytocin, epinephrine, melatonin norepinephrine, DHEA, IL-6, IL-1, IL-4, TNF. Other physiological parameters include eye tracking, pupil tracking, facial expression, thermography, and cognitive and behavioral measures such as stress, anxiety or hyperactivity

The present disclosure provides another method for improving an immune disorder, such as autoimmune disorders, hypersensitivity syndromes, or immune deficiency disorders, in a subject. Such a method comprises determining the level of a physiological parameter that is indicative of immune activity of the subject and predicting dysfunction of the subject's immune system by comparing the determined level of the physiological parameter with a control value. The method further includes placing an ultrasound delivery device into communication with a neural target site that contributes to immune activity if the subject suffers from immune system dysfunction. The method further includes activating the ultrasound delivery device to deliver an ultrasound signal to the neural target site to improve the subject's immune disorder.

With respect to determining a level of a physiological parameter indicative of immune activity, non-limiting physiological parameters include: leukocyte subset ratios, such as the CD4/CD8 ratio; immunoglobulin (antibody) levels or antigen-specific antibody levels (e.g., antibodies directed against specific proteins in the body); NK lysis; granulocyte, neutrophil or monocytic activation; levels of biomarkers of inflammation such as, for example, C-reactive protein; levels of biomarkers of infection such as, for example, procalcitonin; or levels of lymphocytes or leukocytes. The above-described physiological parameters are exemplary. Other physiological parameters can be detected depending on various factors. Such factors include, for example, the specific immune disorder being treated, the subject's age, the body organs affected, the duration or phase of the disorder, and other factors.

Various assays, tests and procedures can be employed to determine the level of a physiological parameter indicative of immune activity. For example, flow cytometry analysis can be used to determine the ratio of leukocyte subsets, such as the CD4/CD8 ratio; blood analysis can be used to determine antibody levels or antigen-specific antibody levels; immune cell function assays (e.g., mitogen stimulation of whole blood cells or lymphocytes with cytokine readouts or proliferation; NK cell lysis assays; or radiological testing, such as T1/T2-weighted imaging of the brain or spinal cord with gadolinium chloride enhancement to identify regions of vascular compromise which are either caused by aberrant inflammation or cause it to develop. The above-described assays, tests and procedures are exemplary. Other techniques can be used to determine the level of a physiological parameter indicative of immune activity depending on various factors. Such factors include, for example, the specific immune disorder being treated, the subject's age, the body organs affected, the duration or phase of the disorder, and other factors.

After determining the level of the physiological parameter, the detected level can be compared to a control value to determine if the detected level is abnormal, thereby indicating that the subject suffers from an immune system dysfunction. Control values can be based upon the level of a corresponding physiological parameter obtained from a control population. The control population can be the general population or a select population. The select population can be a group of healthy subjects, a group of subjects that are at risk of immune system dysfunction, or a group of subjects that suffer from immune system dysfunction. For example, if the level of the physiological parameter is the units of a particular biomarker per ml of blood, the control value can be based upon the units of the particular biomarker per ml of blood in subjects of the general or select population.

As stated above, after comparing the detected level of the physiological parameter with the control value, a determination can be made as to whether the subject has immune system dysfunction. In some instances, an increased level of the detected physiological parameter (as compared to the control value) can be indicated of immune system dysfunction. In other instances, a decreased level of the detected physiological parameter (as compared to the control value) can be indicative of immune system dysfunction. For example, CD4/CD8 ratios are often used as indicators of “adaptive immune health” with a normal ratio of greater than 2.0. Values lower than 2.0 are often used clinically as a diagnostic criteria for various suspected immune diseases including HIV, AIDS, anemia, multiple sclerosis, and chronic infections. A ratio higher 2.0 could indicate the presence of an infection. Furthermore, prolonged elevation of inflammatory markers, such as a C-reactive protein level greater than 5 mg/l, is often associated with exacerbated inflammatory disease; prolonged elevation of markers of infection, such as a procalcitonin level greater than 0.5 ng/ml or a leukocyte level greater than 9 nl, is often associated with exacerbated infectious disease; prolonged suppression of lymphocytes, such as a lymphocyte level less than 1.5/nl or a leukocyte level of less than 4/nl, is often associated with exacerbated immune-suppression or impaired host defense. Other indicators of immune system dysfunction include the presence of pathogenic antibodies; excessive NK lysis; the presence of innate cell respiratory burst function such as granulocyte, neutrophil and monocytic activation; enhanced lesions indicating a break-down of the blood-brain barrier; or the presence of sustained leucopenia, leukocytosis, lymphopenia, lymphocytosis, monocytosis, monopenia, neutrophilia, or neutropenia. The above-described factors and values are exemplary and other factors and values can indicate whether a subject has immune system dysfunction.

### Improving Cancer Symptoms by Neuromodulation of Immune Function

In certain embodiments, a method of improving cancer symptoms in a subject suffering from cancer is provided by delivering an ultrasound signal to a neural target site that contributes to immune activity of a subject. The method further includes activating the ultrasound device to deliver an ultrasound signal to the neural target site and improving the subject's cancer symptoms. By improving the subject's cancer symptoms, the symptoms caused by the cancer are alleviated, the tumor/cancer free interval is prolonged, there is a reduction in tumor/cancer relapse, or the patient's tumor defense mechanisms are enhanced to reduce tumor/cancer treatment associated toxicity (such as reduced cycles of chemotherapy)

Cancer involves an abnormal growth in cells and tissue formation that can spread to other parts of the body. Cancers include all solid tumor and non-solid tumor cancers and can originate in the different organ and tissue systems. For example, the cancer can be breast cancer; cancer of the digestive/gastrointestinal system; endocrine and neuroendocrine cancer; eye cancer; genitourinary cancer; germ cell cancer; bone cancer; gynecologic cancer; head and neck cancer; hematologic/blood cancer; musculoskeletal cancer; neurologic cancer; respiratory/thoracic cancer; skin cancer such as, for example, melanoma; cancer of unknown primary; or AIDS-related cancer.

With respect to the PNS, the neural target site can be a site of the ANS, which includes the sympathetic nervous system (SNS) and the parasympathetic nervous system (PSNS). Such sites include autonomic nerves (including pre- and post-ganglionic fibers of the ANS), autonomic ganglia, and autonomic plexus. Preferably, the neural target site is a part of the SNS, such as a sympathetic ganglion, a sympathetic nerve or a sympathetic plexus. Regarding sympathetic ganglia, the neural target site can be a prevertebral ganglion or a paravertebral ganglion (sympathetic nerve chain ganglion) or the sympathetic trunk. Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus.

It should be appreciated that incorporating the ultrasound device as part of a closed-loop system can include placing the ultrasound device on a mammal at a nerve target site, sensing a physiological parameter associated with immune function, and then activating the ultrasound delivery device to apply an ultrasound signal to adjust application of the ultrasound signal to the nerve target site in response to the sensor signal to modulate immune function. In certain instances, the physiological parameter is associated with a deficient or abnormal immune system such as a hypoactive or hyperactive immune system. In some instances, such physiological parameters can include any characteristic or function associated with immune function, such skin temperature, protein concentrations, heart rate, blood pressure, biomarkers of the immune system or autonomic nervous system, electrochemical gradients, electrolytes, laboratory values, body temperature, and vital signs.

Sensors to measure physiological parameters can be external of the patient's body, or on the patient's body. The parameters can be measured, for example, from blood, saliva, or sweat biomarkers. Non-limiting examples of physiological parameters include vital signs; EEG, EMG; FNIRS; heart rate variability; immune and hormonal markers, IL-6, TNF, other inflammatory mediators; sedimentation rate such as ESR for example, CRP, CBC and differential ratios of cell; circadian rhythms; and other means of feedback linked to stimulation modulation. Physiological parameters that are biomarkers include but are not limited to BDNF, Neuropeptide Y, cortisol, orexin, oxytocin, epinephrine, melatonin norepinephrine, DHEA, IL-6, IL-1, IL-4, TNF. Other physiological parameters include eye tracking, pupil tracking, facial expression, thermography, and cognitive and behavioral measures such as stress, anxiety or hyperactivity.

The present disclosure provides another method for improving cancer symptoms in a subject. Such a method comprises determining the level of a physiological parameter that is indicative of immune activity of the subject and predicting dysfunction of the subject's immune system by comparing the determined level of the physiological parameter with a control value. The method further includes placing an ultrasound delivery device into communication with a neural target site that contributes to immune activity if the subject suffers from immune system dysfunction. The method further includes activating the ultrasound delivery device to deliver an ultrasound signal to the neural target site to improve the subject's cancer symptoms.

With respect to determining a level of a physiological parameter indicative of immune activity, non-limiting physiological parameters include: leukocyte subset ratios, such as the CD4/CD8 ratio; immunoglobulin (antibody) levels or antigen-specific antibody levels (e.g., antibodies directed against specific proteins in the body); NK lysis; granulocyte, neutrophil or monocytic activation; levels of biomarkers of inflammation such as, for example, C-reactive protein; levels of biomarkers of infection such as, for example, procalcitonin; or levels of lymphocytes or leukocytes. The above-described physiological parameters are exemplary. Other physiological parameters can be detected depending on various factors. Such factors include, for example, the specific cancer being treated, the subject's age, the body organs affected, the duration or phase of the cancer, and other factors.

Various assays, tests and procedures can be employed to determine the level of a physiological parameter indicative of immune activity. For example, flow cytometry analysis can be used to determine the ratio of leukocyte subsets, such as the CD4/CD8 ratio; blood analysis can be used to determine antibody levels or antigen-specific antibody levels; immune cell function assays (e.g., mitogen stimulation of lymphocytes with cytokine readouts or proliferation; NK cell lysis assays; or radiological testing, such as T1/T2-weighted imaging of the brain or spinal cord with gadolinium chloride enhancement to identify regions of vascular compromise which are either caused by aberrant inflammation or cause it to develop. The above-described assays, tests and procedures are exemplary. Other techniques can be used to determine the level of a physiological parameter indicative of immune activity depending on various factors. Such factors include, for example, the specific cancer being treated, the subject's age, the body organs affected, the duration or phase of the cancer, and other factors.

After determining the level of the physiological parameter, the detected level can be compared to a control value to determine if the detected level is abnormal, thereby indicating that the subject suffers from an immune system dysfunction. Control values can be based upon the level of a corresponding physiological parameter obtained from a control population. The control population can be the general population or a select population. The select population can be a group of healthy subjects, a group of subjects that are at risk of immune system dysfunction, or a group of subjects that suffer from immune system dysfunction. For example, if the level of the physiological parameter is the units of a particular biomarker per ml of blood, the control value can be based upon the units of the particular biomarker per ml of blood in subjects of the general or select population.

As stated above, after comparing the detected level of the physiological parameter with the control value, a determination can be made as to whether the subject has immune system dysfunction. In some instances, an increased level of the detected physiological parameter (as compared to the control value) can be indicated of immune system dysfunction. In other instances, a decreased level of the detected physiological parameter (as compared to the control value) can be indicative of immune system dysfunction. For example, CD4/CD8 ratios are often used as indicators of “adaptive immune health” with a normal ratio of greater than 2.0. Values lower than 2.0 are often used clinically as a diagnostic criteria for various suspected immune diseases including HIV, AIDS, anemia, multiple sclerosis, and chronic infections. A ratio higher 2.0 could indicate the presence of an infection. Furthermore, prolonged elevation of inflammatory markers, such as a C-reactive protein level greater than 5 mg/l, is often associated with exacerbated inflammatory disease; prolonged elevation of markers of infection, such as a procalcitonin level greater than 0.5 ng/ml or a leukocyte level greater than 9 nl, is often associated with exacerbated infectious disease; prolonged suppression of lymphocytes, such as a lymphocyte level less than 1.5/nl or a leukocyte level of less than 4/nl, is often associated with exacerbated immune-suppression or impaired host defense. Other indicators of immune system dysfunction include the presence of pathogenic antibodies; excessive NK lysis; the presence of innate cell respiratory burst function such as granulocyte, neutrophil and monocytic activation; enhanced lesions indicating a break-down of the blood-brain barrier; or the presence of sustained leucopenia, leukocytosis, lymphopenia, lymphocytosis, monocytosis, monopenia, neutrophilia, or neutropenia. The above-described factors and values are exemplary and other factors and values can indicate whether a subject has immune system dysfunction.

### Improving Infections by Neuromodulation of Immune Function

Methods as described herein with respect to improving an infection can also apply to preventing an infection.

As described herein, a patient's infection is caused by an infectious agent. Such infectious agents include prions; viruses; bacteria; bacteriophages; plasmids; transposons; fungi; protozoa; helminthes; and parasites (including endoparasites and ectoparsites). Viral infections include transient infections, chronic latent infections, chronic productive infections, and transforming infections. Non-limiting examples of transient viral infections are measles, mumps, poliovirus infection, west nile virus, and viral hemorrhagic fevers. Non-limiting examples of chronic viral infections are herpes simplex virus infections, cytomegalovirus infections, and varicella zoster virus infections. Non-limiting examples of chronic productive viral infections are hepatitis B viral infections. Non-limiting examples of transforming viral infections are Epstein-Barr virus infections, and human papillovirus infections. Bacterial infections include gram-positive bacterial infections, gram-negative bacterial infections, myobacterial infections, spirochetes infections, anaerobic bacterial infections, and obligate intracellular bacterial infections. Non-limiting examples of gram positive bacterial infections are staphylococcal infections, streptococcal infections, diphtheria, listeriosis, anthrax, and nocardia infection. Non-limiting examples of gram-negative bacterial infections are neisserial infections, whooping cough, pseudomonas infection, plague, chancroid, and granuloma inguinale. Non-limiting examples of spirochete infections are syphilis, relapsing fever, and Lyme disease. Non-limiting examples of anaerobic bacterial infections are abscesses and clostridial infections. Non-limiting examples of obligate intracellular bacterial infections are chlamydial infections and rickettsial infections. Fungal infections include yeast and mold infections. Non-limiting examples of yeast infections are candidiasis and cryptococcosis. Non-limiting examples of mold infections are aspergillosis and zygomycosis. Parasitic infections include protozoan infections and metazoan infections. Non-limiting examples of protozoan infections are malaria, babesiosis, leishmaniasis, African trypanosomiasis, and Chagas disease. Non-limiting examples of metazoan infections are strongyloidiasis, tapeworms, cysticercosis, hydatid disease, trichinosis, schistosomiasis, lymphatic filariasis, and onchocerciasis. Other types of infections are Ebola, avian flu, chicken pox, adenovirus, rhinovirus infections, coxsackie, herpes, and HIV infections.

The neural target site can be a PNS structure anatomically relevant to maladaptive sympathetic reflexes or disturbances. Preferably, the neural target site innervates an endocrinological or lymphatic tissue or organ involved in the immune response of the patient. In certain embodiments, the neural target site is a neural target site of the ANS. For example, the neural target site can be a celiac ganglion, a superior mesenteric ganglion, an aorticorenal ganglion, a renal plexus, a inferior mesenteric ganglion, a superior hypogastric ganglion, a lumbar plexus, a celiac plexus, a splenic nerve, a sympathetic trunk, a splanchnic nerve, an intrinsic nervous system of an organ and their input and output nervous system structures.

Further with respect to the neural target site being a site of the ANS, the ANS includes the sympathetic nervous system (SNS) and the parasympathetic nervous system (PSNS). Such sites include autonomic nerves (including pre- and post-ganglionic fibers of the ANS), autonomic ganglia, and autonomic plexus. Preferably, the neural target site is a part of the SNS, such as a sympathetic ganglion, a sympathetic nerve or a sympathetic plexus. Regarding sympathetic ganglia, the neural target site can be a prevertebral ganglion or a paravertebral ganglion (sympathetic nerve chain ganglion) as well as the sympathetic trunk. Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus.

It should be appreciated that incorporating an ultrasound delivery device as part of a closed-loop system can include placing an ultrasound delivery device on a mammal at a nerve target site, sensing a physiological parameter associated with immune function, and then activating the ultrasound delivery device to apply a therapy signal to adjust application of the ultrasound signal to the nerve target site in response to the sensor signal to modulate immune function. In certain instances, the physiological parameter is associated with a deficient or abnormal immune system such as a hypoactive or hyperactive immune system. In some instances, such physiological parameters can include any characteristic or function associated with immune function, such skin temperature, protein concentrations, heart rate, blood pressure, biomarkers of the immune system or autonomic nervous system, electrochemical gradients, electrolytes, laboratory values, body temperature, and vital signs. Sensors to measure physiological parameters can be external of the patient's body, or on the patient's body. The parameters can be measured, for example, from blood, saliva, or sweat biomarkers. Non-limiting examples of physiological parameters include vital signs; EEG, EMG; FNIRS; heart rate variability; immune and hormonal markers, IL-6, TNF, other inflammatory mediators; sedimentation rate such as ESR for example, CRP, CBC and differential ratios of cell; circadian rhythms; and other means of feedback linked to stimulation modulation. Physiological parameters that are biomarkers include but are not limited to BDNF, Neuropeptide Y, Cortisol, orexin, oxytocin, epinephrine, melatonin norepinephrine, DHEA, IL-6, IL-1, IL-4, TNF. Other physiological parameters include eye tracking, pupil tracking, facial expression, thermography, and cognitive and behavioral measures such as stress, anxiety or hyperactivity.

The present disclosure provides another method for improving an infection in a subject. Such a method comprises determining the level of a physiological parameter that is indicative of immune activity of the subject and predicting dysfunction of the subject's immune system by comparing the determined level of the physiological parameter with a control value. The method further includes placing an ultrasound delivery device into communication with a neural target site that contributes to immune activity if the subject suffers from immune system dysfunction. The method further includes activating the ultrasound delivery device to deliver an ultrasound signal to the neural target site to improve the subject's infection.

With respect to determining a level of a physiological parameter indicative of immune activity, non-limiting physiological parameters include: leukocyte subset ratios, such as the CD4/CD8 ratio; immunoglobulin (antibody) levels or antigen-specific antibody levels (e.g., antibodies directed against specific proteins in the body); NK lysis; granulocyte, neutrophil or monocytic activation; levels of biomarkers of inflammation such as, for example, C-reactive protein; levels of biomarkers of infection such as, for example, procalcitonin; or levels of lymphocytes or leukocytes. The above-described physiological parameters are exemplary. Other physiological parameters can be detected depending on various factors. Such factors include, for example, the specific infection being treated, the subject's age, the body organs affected, the duration or phase of the infection, and other factors.

Various assays, tests and procedures can be employed to determine the level of a physiological parameter indicative of immune activity. For example, flow cytometry analysis can be used to determine the ratio of leukocyte subsets, such as the CD4/CD8 ratio; blood analysis can be used to determine antibody levels or antigen-specific antibody levels; immune cell function assays (e.g., mitogen stimulation of lymphocytes with cytokine readouts or proliferation; NK cell lysis assays; or radiological testing, such as T1/T2-weighted imaging of the brain or spinal cord with gadolinium chloride enhancement to identify regions of vascular compromise which are either caused by aberrant inflammation or cause it to develop. The above-described assays, tests and procedures are exemplary. Other techniques can be used to determine the level of a physiological parameter indicative of immune activity depending on various factors. Such factors include, for example, the specific infection being treated, the subject's age, the body organs affected, the duration or phase of the infection, and other factors.

After determining the level of the physiological parameter, the detected level can be compared to a control value to determine if the detected level is abnormal, thereby indicating that the subject suffers from an immune system dysfunction. Control values can be based upon the level of a corresponding physiological parameter obtained from a control population. The control population can be the general population or a select population. The select population can be a group of healthy subjects, a group of subjects that are at risk of immune system dysfunction, or a group of subjects that suffer from immune system dysfunction. For example, if the level of the physiological parameter is the units of a particular biomarker per ml of blood, the control value can be based upon the units of the particular biomarker per ml of blood in subjects of the general or select population.

As stated above, after comparing the detected level of the physiological parameter with the control value, a determination can be made as to whether the subject has immune system dysfunction. In some instances, an increased level of the detected physiological parameter (as compared to the control value) can be indicated of immune system dysfunction. In other instances, a decreased level of the detected physiological parameter (as compared to the control value) can be indicative of immune system dysfunction. For example, CD4/CD8 ratios are often used as indicators of “adaptive immune health” with a normal ratio of greater than 2.0. Values lower than 2.0 are often used clinically as a diagnostic criteria for various suspected immune diseases including HIV, AIDS, anemia, multiple sclerosis, and chronic infections. A ratio higher 2.0 could indicate the presence of an infection. Furthermore, prolonged elevation of inflammatory markers, such as a C-reactive protein level greater than 5 mg/l, is often associated with exacerbated inflammatory disease; prolonged elevation of markers of infection, such as a procalcitonin level greater than 0.5 ng/ml or a leukocyte level greater than 9 nl, is often associated with exacerbated infectious disease; prolonged suppression of lymphocytes, such as a lymphocyte level less than 1.5/nl or a leukocyte level of less than 4/nl, is often associated with exacerbated immune-suppression or impaired host defense. Other indicators of immune system dysfunction include the presence of pathogenic antibodies; excessive K lysis; the presence of innate cell respiratory burst function such as granulocyte, neutrophil and monocytic activation; enhanced lesions indicating a break-down of the blood-brain barrier; or the presence of sustained leucopenia, leukocytosis, lymphopenia, lymphocytosis, monocytosis, monopenia, neutrophilia, or neutropenia. The above-described factors and values are exemplary and other factors and values can indicate whether a subject has immune system dysfunction.

### Post-Traumatic Stress Disorder

A patient diagnosed with PTSD can refer to having a diagnosis of at least one sign, symptom, or symptom cluster indicative of PTSD. Non-limiting examples of such traumatic events can include military combat, terrorist incidents, physical assault, sexual assault, motor vehicle accidents, and natural disasters.

The Diagnostic and Statistical Manual of Mental Disorders-IV-Text revised (DSM-IV-TR), a handbook for mental health professionals that lists categories of mental disorders and the criteria, classifies PTSD as an anxiety disorder. According to the DSM-IV-TR, a PTSD diagnosis can be made if:
1. the patient experienced, witnessed, or was confronted with an event or events that involved actual or threatened death or serious injury, or a threat to the physical integrity of self or others and the response involved intense fear, helplessness, or horror;
2. as a consequence of the traumatic event, the patient experiences at least one re-experiencing/intrusion symptom, three avoidance/numbing symptoms, and two hyperarousal symptoms, and the duration of the symptoms is for more than 1 month; and
3. the symptoms cause clinically significant distress or impairment in social, occupational, or other important areas of functioning.

In some instances, if the patient's disorder fulfills DSM-IV-TR criteria, the patient is diagnosed with PTSD. In other instances, if the patient has at least one sign, symptom, or symptom cluster of PTSD, the patient is diagnosed with PTSD. In further instances, a scale can be used to measure a sign, symptom, or symptom cluster of PTSD, and PTSD can be diagnosed on the basis of the measurement using that scale. In some instances, a “score” on a scale can be used to diagnose or assess a sign, symptom, or symptom cluster of PTSD. In other instances, a “score” can measure at least one of the frequency, intensity, or severity of a sign, symptom, or symptom cluster of PTSD.

The term scale can refer to a method to measure at least one sign, symptom, or symptom cluster of PTSD in a patient. In some instances, a scale may be an interview or a questionnaire. Non-limiting examples of scales include Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IBS), Impact of Event Scale-Revised (IES-R), Clinical Global Impression Scale (CGI), Clmical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Asb erg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

The terms “sign” and “signs” can refer to objective findings of a disorder (e.g., PTSD). In some instances, a sign can be a physiological manifestation or reaction of a disorder (e.g., PTSD). For example, a sign may include heart rate and rhythm, body temperature, pattern and rate of respiration, papillary changes and blood pressure. In other instances, signs can be associated with, or indicative of, symptoms.

The terms symptom and symptoms can refer to subjective indications that characterize a disorder. Symptoms of PTSD may refer to, for example, recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In some instances, the physiological reactivity manifests in at least one of abnormal respiration, abnormal cardiac rate of rhythm, abnormal blood pressure, abnormal function of a special sense, and abnormal function of sensory organ. In other instances, restricted range of effect characterized by diminished or restricted range or intensity of feelings or display of feelings can occur and a sense of a foreshortened future can manifest in thinking that one will not have a carcer, marriage, children, or a normal life span. In further instances, children and adolescents may have symptoms of PTSD, such as disorganized or agitated behavior, repetitive play that expresses aspects of the trauma, frightening dreams which lack recognizable content, and trauma-specific reenactment.

The term symptom cluster” can refer to a set of signs, symptoms, or a set of signs and symptoms that are grouped together because of their relationship to each other or their simultaneous occurrence. In some instances, for example, PTSD is characterized by three symptom clusters: re-experiencing/intrusion; avoidance/numbing; and hyperarousal.

The term re-experiencing/intrusion can refer to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In some instances, the physiological reactivity can manifest in at least one of abnormal respiration, abnormal cardiac rate of rhythm, abnormal blood pressure, abnormal function of a special sense, and abnormal function of sensory organ.

The term avoidance/numbing can refer to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

The term hyperarousal can refer to at least one of difficulty falling or staying asleep, irritability or outbursts of anger, difficulty concentrating, hypervigilance, and exaggerated startle response.

Target ANS sites include, for example, a stellate ganglion, a cervical ganglion, or combinations thereof. Target feedback parameters include parameters as disclosed above.

### Fibromyalgia

Fibromyalgia can refer to chronic and frequently difficult-to-manage pain in muscle and soft tissues surrounding the joints of unknown etiology characterized by widespread pain, abnormal pain processing, sleep disturbance, fatigue and psychological distress. In some instances, the terms “fibromyalgia,” “fibromyalgia syndrome,” “myofascial syndrome” and “fibromyalgia and related syndromes” can be used interchangeably and refer to a chronic pain disorder characterized by one or more of the following: pain including allodynia (painful response to a stimulus that is not usually painful) and hyperalgesia (exaggerated response to a stimulus that is usually only mildly painful); a series of regional pains, such as non-cardiac chest pain, dyspepsia, headache, abdominal cramping (irritable bowel syndrome), temporomandibular pain and chronic pelvic pain; stiffness; chronic aching in multiple areas of the musculoskeletal system; fatigue; poor sleep; tender points; cognitive difficulties with attention and memory; weight fluctuations; allergic symptoms (e.g., nasal congestion); hypersensitivity to environmental stimuli (e.g., odors, bright lights, loud noises) and medications; syncope; shortness of breath; and urinary frequency and urgency. In other instances, the 2010 criteria set forth by the American College of Rheumatology can be used to diagnose and/or assess severity of fibromyalgia in a subject. For instance, diagnosis can be based on: a Widespread Pain Index (WPI)>7 and a symptom severity scale (SS)>5 or WPI 3-6 and SS>9; symptoms having been present at a similar level for at least 3 months; and/or the subject not having a disorder that would otherwise explain the pain. The term “fibromyalgia” can also refer to primary or secondary fibromyalgia. “Primary fibromyalgia” can refer to fibromyalgia in which the only rheumatic disorder the subject is suffering from is fibromyalgia, whereas the term “secondary fibromyalgia” can refer to fibromyalgia that occurs in conjunction with another diagnosed rheumatic disorder. In some instances, “fibromyalgia and related syndromes” can include fibromyalgia-related fatigue syndromes, pain syndromes, and sleep disturbances.

Examples of ANS nerve targets on which an ultrasound delivery device may be placed in communication with can include, but are not limited to, a sympathetic chain ganglion, an efferent of a sympathetic chain ganglion, or an afferent of a sympathetic chain ganglion. In some instances, the sympathetic chain ganglion can be selected from the group consisting of a superior cervical ganglion, a middle cervical ganglion, an inferior cervical ganglion, and a stellate ganglion. Additional examples of ANS nerve targets into which a therapy delivery device may be placed into electrical communication can include one or more parasympathetic nerves or nerve structures. In one example, an ultrasound device can be placed in communication with a parasympathetic nerve or nerve structure. Feedback parameters includes those disclosed above.

### Uterine Function

Methods and system for modulating uterine function in a mammal are also provided herein. As discussed above, the ANS is responsible for regulating the intrinsic function and balance (or homeostasis) of each body organ. The uterus, for example, is innervated by sympathetic and parasympathetic fibers of the ANS. In pregnancy, there is a progressive sympathetic denervation of the uterus, which leaves the myometrium sensitive to circulating norepinephrine (contractile response) and beta-2-adrenergic agents (relaxation response). As described in detail below, the present disclosure advantageously provides devices, systems, and methods for precise and selective control of the ANS to modulate uterine function. By use of such devices, systems and methods, the present disclosure can, in one example, reduce pre-term labor contractions and thereby reduce subsequent pre-term births.

In general, methods of the present disclosure can include the steps of providing an ultrasound delivery device; placing the ultrasound delivery device in communication with a nerve target of a mammal that is associated with uterine function; and activating the ultrasound delivery device to deliver an ultrasound signal to an ANS target site in an amount and for a time sufficient to effect a change in sympathetic and/or parasympathetic activity in the mammal and thereby modulate uterine function.

Ultrasound delivery to an ANS target site may affect central motor output, nerve conduction, neurotransmitter release, synaptic transmission, and/or receptor activation. For example, at least a portion of the ANS may be modulated to alter, shift, or change parasympathetic activity from a first state to a second state, where the second state is characterized by an increase or decrease in parasympathetic activity relative to the first state. Alternatively, at least a portion of the ANS may be modulated to alter, shift, or change sympathetic activity from a first state to a second state, where the second state is characterized by an increase or decrease in sympathetic activity relative to the first state.

It will be appreciated that delivering ultrasound for example, to a target nerve can modulate the ANS in a desirable combination of ways, such as increasing both parasympathetic and sympathetic activity, increasing parasympathetic activity while decreasing sympathetic function, decreasing both parasympathetic and sympathetic activity, and decreasing parasympathetic activity while increasing sympathetic activity.

Examples of nerve targets upon which an ultrasound delivery device may be placed in communication with can include, but are not limited to a sympathetic chain ganglion including a thoracic sympathetic chain ganglion, a cervical chain ganglion, a lower cervical chain ganglion, an inferior cervical chain ganglion, a stellate ganglion; a hypogastric plexus, an inferior hypogastric plexus, a sacral plexus, an inferior mesenteric ganglion, a superior mesenteric ganglion, a lesser splanchnic nerve, or a sacral splanchnic nerve.

In certain aspects, methods and systems can involve delivering an ultrasound signal to reduce or prevent pre-term labor in the mammal. A physiological parameter associated with pre-term labor can be sensed and a sensor signal can be generated based on the physiological parameter. An ultrasound delivery device can adjust application of an ultrasound signal to an ANS target site in response to the sensor signal to prevent or mitigate the pre-term labor. In certain aspects, methods and systems can involve delivering an ultrasound signal to stimulate labor in a post-term pregnant mammal. A physiological parameter associated with post-term labor can be sensed and a sensor signal can be generated based on the physiological parameter. An ultrasound delivery device can adjust application of an ultrasound signal to an ANS target site in response to the sensor signal to stimulate post-term labor.

In some instances, physiological parameters can serve as feedback in the ultrasound therapy and can include a characteristic or function associated with uterine function, such skin temperature, protein concentrations (e.g., fetal fibronectin), uterus and other pressure sensing, electrochemical gradients, hormones (e.g., catecholamines), electrolytes, laboratory values, body temperature, vital signs, changes in cervical length (e.g., as measured by ultrasound), the recorded combination of uterine contractions and the release of fetal fibronectin, or other signs and biomarkers associated with uterine function. In other instances, an ultrasound delivery device can be operably linked to an external tocodynomometer and/or a myometrial electrode or sensor (e.g., configured to record uterine contractions) to form a closed-loop system.

### Metabolic Disorders

In certain aspects, methods and systems are provided herein that include activating an ultrasound delivery device to deliver an ultrasound signal to an ANS target site and improving the subject's metabolic syndrome. By improving the subject's metabolic syndrome, the symptoms caused by the metabolic syndrome are alleviated, the number of symptoms is reduced, or the severity of symptoms is reduced. Methods and systems as described herein can also be used to prevent metabolic syndrome, for example, in patients at risk of developing metabolic syndrome. Such patients include patients with genetic risk factors or other risk factors for obesity.

Metabolic syndrome can be a disorder that affects glucose utilization and storage, blood lipid composition, or liver enzyme synthesis. Metabolic syndrome is characterized by abdominal obesity, elevated blood pressure, elevated fasting plasma glucose, insulin resistance, high serum triglycerides, low high-density lipoprotein levels, or combinations thereof. In particular, metabolic syndrome is the name for a group of risk factors that raises a subject's risk for heart disease and other health problems, such as diabetes and stroke. It includes different symptoms including, but not limited to, five conditions described below that are generally considered metabolic risk factors. At least three metabolic risk factors are generally required to be diagnosed with metabolic syndrome. The first condition is abdominal obesity. The second condition is a high triglyceride level or if the subject is on medicine to treat high triglycerides. The third condition is a low HDL cholesterol level or if the subject is on medication to treat low HDL cholesterol. The fourth condition is high blood pressure or if the subject is on medication to treat high blood pressure. The fifth condition is a high fasting blood sugar level or if the subject is on medication to treat a high blood sugar level.

In terms of targets sites, the neural target site can be a site of the ANS, which includes the sympathetic nervous system (SNS) and the parasympathetic nervous system (PSNS). Such sites include autonomic nerves (including pre- and post-ganglionic fibers of the ANS), autonomic ganglia, and autonomic plexus. Preferably, the neural target site is a part of the SNS, such as a sympathetic ganglion, a sympathetic nerve or a sympathetic plexus. Regarding sympathetic ganglia, the neural target site can be a prevertebral ganglion or a paravertebral ganglion (sympathetic nerve chain ganglion) or the sympathetic trunk. Examples of paravertebral ganglia include a cervical ganglion, a thoracic ganglion, a lumbar ganglion, and a sacral ganglion. Cervical ganglia include a superior cervical ganglion, a middle cervical ganglion, and an inferior cervical ganglion (or a stellate ganglion). Examples of prevertebral ganglia include a celiac ganglion, an aorticorenal ganglion, a superior mesenteric ganglion, and an inferior mesenteric ganglion. Regarding sympathetic nerves, the neural target site can be a splanchnic nerve, including a greater, lesser, and least splanchnic nerve. Regarding autonomic plexus, the neural target site can be a superior hypogastric plexus or a pulmonary plexus. In certain embodiments, the neural target site is a neural target site of the ANS. For example, the neural target site can be a celiac ganglion, a superior mesenteric ganglion, an aorticorenal ganglion, a renal plexus, a inferior mesenteric ganglion, a superior hypogastric ganglion, a lumbar plexus, a celiac plexus, a splenic nerve, a sympathetic trunk, a splanchnic nerve and their input and output nervous system structures

It should be appreciated that incorporating an ultrasound delivery device as part of a closed-loop system can include placing an ultrasound delivery device on a mammal at a ANS target site, sensing a physiological parameter associated with metabolic function, and then activating the ultrasound delivery device to apply a therapy signal to adjust application of the ultrasound signal to the ANS target site in response to the sensor signal to modulate metabolic function. In certain instances, the physiological parameter is associated with a deficient or abnormal metabolic system. In some instances, such physiological parameters can include any characteristic or function associated with metabolic function.

Sensors to measure physiological parameters can be external of the patient's body, or on the patient's body. The parameters can be measured, for example, from blood, saliva, or sweat biomarkers. Non-limiting examples of physiological parameters include vital signs; EEG, EMG; FNIRS; heart rate variability; immune and hormonal markers, IL-6, TNF, other inflammatory mediators; sedimentation rate such as ESR for example, CRP, CBC and differential ratios of cell; circadian rhythms; and other means of feedback linked to stimulation modulation. Physiological parameters that are biomarkers include but are not limited to BDNF, Neuropeptide Y, Cortisol, orexin, oxytocin, epinephrine, melatonin norepinephrine, DHEA, IL-6, IL-1, IL-4, TNF. Other physiological parameters include eye tracking, pupil tracking, facial expression, thermography, and cognitive and behavioral measures such as stress, anxiety or hyperactivity.

### Anxiety and Anxiety Associated Disorders and Autism

As used herein, the term anxiety disorder can refer to a dysfunctional state of fear and anxiety, e.g., fear and anxiety that is out of proportion to a stressful situation or the anticipation of a stressful situation. In some instances, an anxiety disorder can be any one or combination of generalized anxiety disorders, such as depression, panic disorder, panic disorder with agoraphobia, agoraphobia, social anxiety disorder, a generalized stress disorder, a stress-induced anxiety disorder, addiction (e.g., alcoholism), an eating disorder and obsessive-compulsive disorder. As used herein, the term eating disorder can refer to any disease or condition that is characterized, at least in part, by obsession with body weight and food as well as abnormal compulsions to avoid eating or uncontrollable impulses to consume abnormally large amounts of food. Eating disorders may affect not only the social well-being, but also the physical well-being of sufferers. Eating disorders can be caused by, a symptom of, co-morbid, or otherwise associated with one or more anxiety disorders, mood disorders, neurobehavioral disorders (e.g., psychotic or psychiatric disorders, such as schizophrenia), and/or abnormalities in Body Mass Index, metabolic rate, etc. Non-limiting examples of eating disorders can include anorexia nervosa, bulimia, binge or compulsive eating, obesity, cachexia and wasting syndromes. As used herein, the term anxiety-associated disorder can refer to a dysfunctional or abnormal psychological and physiological state characterized by cognitive, somatic, emotional, and behavioral components that is related to, caused at least in part by, or correlated with the presence of a co-morbid disease, disorder or condition, such as an eating disorder, autism or other disorder in the autism spectrum. In some instances, these components combine to create the painful feelings typically recognized as anger, fear, apprehension or worry. As used herein, the term autism can refer to any disease or disorder characterized by impaired social interaction and communication, and by restricted and repetitive behavior. In some instances, autism can refer to one of three recognized disorders in the autism spectrum, the other two being Asperger syndrome and pervasive developmental disorder.

The ANS, and in particular the SNS, plays a crucial role in patients with anxiety or anxiety-associated disorders. Part of the proposed pathophysiology of certain anxiety or anxiety-associated disorders is that systemic autonomic responses to certain internal external stimuli (e.g., stress) feed back to increase the level of emotional arousal and anxiety. As described in detail below, the present disclosure advantageously provides, in some instances, ultrasound delivery devices for modulating the descending nerve signals from the brain through the SNS to peripheral tissues (e.g., cardiac, gastric, vascular, immunological, and other related tissues/organs), as well as ascending signals into the CNS to effectively normalize or regulate the ANS (e.g., the SNS). By employing such devices, systems and methods, the present disclosure can improve anxiety or anxiety-associated disorders by, for example, replacing conventional treatment modalities, such as pharmacological treatments.

Examples of ANS targets were an ultrasound delivery device may be placed in communication with can include, but are not limited to, tissues of the SNS or the PNS. In some instances, autonomic nervous tissue targets onto which an ultrasound delivery device may be placed into communication with can include a sympathetic chain ganglion, an efferent of a sympathetic chain ganglion, or an afferent of a sympathetic chain ganglion. In other instances, the sympathetic chain ganglion can be a cervical sympathetic ganglion, a thoracic sympathetic ganglion, or a stellate ganglion. Examples of cervical sympathetic ganglia can include an upper cervical sympathetic ganglion, a middle cervical sympathetic ganglion, or a lower cervical sympathetic ganglion. Examples of thoracic sympathetic ganglia can include a T1 sympathetic ganglia, a T2 sympathetic ganglia, a T3 sympathetic ganglia, a T4 sympathetic ganglia, a T6 sympathetic ganglia, or a T7 sympathetic ganglia.

It should be appreciated that implementing an ultrasound delivery device as part of a closed-loop system can include placing an ultrasound delivery device on a subject at an autonomic nervous tissue target, sensing a physiological parameter associated with an anxiety or anxiety-associated disorder, and then activating the ultrasound delivery device to apply an ultrasound signal to adjust application of the electrical signal to the autonomic nervous tissue target in response to the sensor signal. In some instances, such physiological parameters can include any characteristic, sign, symptom, or function associated with an anxiety or anxiety-associated disorder, such as a chemical moiety or nerve activity (e.g., electrical activity). Examples of such chemical moieties and nerve activities can include the activity of autonomic ganglia (or an autonomic ganglion), the activity of a spinal cord segment or spinal nervous tissue associated therewith, protein concentrations, electrochemical gradients, hormones (e.g., Cortisol), neuroendocrine markers, such as corticosterone, norepinephrine and melatonin, electrolytes, laboratory values, vital signs (e.g., blood pressure), markers of locomotor activity, cardiac markers (e.g., EKG RR intervals), or other signs and biomarkers associated with an anxiety or anxiety-associated disorder.

The disclosed systems and methods can be used to initially treat a patient, screen or select a patient for therapy, and adjust follow up ultrasound therapy sessions. Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure. Further, unless otherwise specified, none of the steps of the methods of the present disclosure are confined to any particular order of performance. Furthermore, all references cited herein are incorporated by reference in their entirety.

What is claimed is:

1. A method of improving a medical condition in a patient in need thereof comprising:
delivering a focused ultrasound neuromodulation signal to a neural target site of the patient's autonomic nervous system, the ultrasound signal having a mechanical index of between about 0.1 to about 5.0 and/or an acoustic pressure of between about 0.1 MPa to about 2.5 MPa, the medical condition comprising chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence or binge eating; and
improving the patient's medical condition.

2. The method of claim 1, further comprising:
screening a patient to determine if the focused ultrasound neuromodulation signal is appropriate for the patient to treat the medical condition;
monitoring the patient to measure a plurality of parameters for the patient and detect or predict an onset of symptoms associated with the disorder from the plurality of parameters if the focused ultrasound neuromodulation signal has been determined to be appropriate for the patient;
selecting a location within the neural target site according to at least one of the parameters when an onset of symptoms has been detected or predicted;
selecting a dosage parameter associated with the focused ultrasound neuromodulation signal according to at least one of the plurality of parameters, the focused ultrasound neuromodulation signal being delivered with the selected dosage parameter; and
measuring the plurality of parameters after the focused ultrasound neuromodulation signal is delivered to determine an effectiveness of the focused ultrasound neuromodulation signal.

3. The method of claim 2, wherein the location is a first location and the method further comprises:
determining, from the plurality of parameters, that the focused ultrasound neuromodulation signal is not effective in treating the medical condition;
selecting a second location within the neural target site; and
delivering the focused ultrasound neuromodulation signal to the second location.

4. The method of claim 2, wherein the dosage parameter is a first dosage parameter and the method further comprises:
determining, from the plurality of parameters, that the focused ultrasound neuromodulation signal is not effective in treating the medical condition;
selecting a second dosage parameter according to at least one of the plurality of parameters; and
delivering the focused ultrasound neuromodulation signal using the second dosage parameter.

5. The method of claim 2, wherein the method further comprises:
determining, from the plurality of parameters, that the focused ultrasound neuromodulation signal is effective in treating the medical condition;
predicting an onset of symptoms for the patient from the plurality of parameters; and
delivering the focused ultrasound neuromodulation signal to the location within the neural target site in response to predicting the onset of symptoms.

6. The method of claim 2, wherein the method further comprises:
determining, from the plurality of parameters, that the focused ultrasound neuromodulation signal is not effective in treating the medical condition; and
selecting an alternative therapy for the medical condition.

7. A computer program comprising instructions, which, when the program is executed by a processor causes the step of:
controlling an ultrasound device to deliver a focused ultrasound neuromodulation signal to a neural site of the autonomic nervous system, the ultrasound signal having a mechanical index of between about 0.1 to about 5.0 and/or an acoustic pressure of between about 0.1 MPa to about 2.5 MPa and to a site of the ANS sufficient to improve chronic or refractory rhinitis, chronic rhinosinusitis, autonomic instability/autonomic dysfunction, functional gastrointestinal disorders, inflammatory disorders, immune disorders (including improving cancer and infections by neuromodulation of immune function), complex regional pain syndrome, post-traumatic stress disorder (PTSD), anxiety and anxiety-associated disorders, autism, fibromyalgia, uterine function, metabolic disorder, urinary incontinence, or binge eating.
